(19) United States
(12) Patent Application Publication
(10) Pub. No.: US 2026/0022375 A1
(43) Pub. Date: Jan. 22, 2026

(54) METHODS AND COMPOSITIONS FOR SELECTIVELY MODULATING GENE EXPRESSION IN MEGAKARYOCYTES AND PLATELETS

(71) Applicant: Thomas Jefferson University, Philadelphia, PA (US)

(72) Inventors: Lawrence Elliot Goldfinger, Philadelphia, PA (US); Jeremy Garfield Takashi Wurtzel, New Jersey, PA (US)

(21) Appl. No.: 18/996,606

(22) PCT Filed: Jul. 19, 2023
(86) PCT No.: PCT/US2023/070508

§ 371 Date: Jan. 17, 2025

Related U.S. Application Data
(60) Provisional application No. 63/391,002, filed on Jul. 21, 2022.
(60) Provisional application No. 63/439,365, filed on Jan. 17, 2023.
(60) Provisional application No. 63/522,839, filed on Jun. 23, 2023.

(51) Int. Cl. C12N 15/113; A61P 7/02

(52) U.S. Cl. CPC C12N 15/113; A61P 7/02; C12N 2310/14; C12N 2310/141; C12N 2310/321; C12N 2310/322; C12N 2310/3231; C12N 2320/32

(57) ABSTRACT

A method of selectively modulating gene expression of one or more target genes in megakaryocytes, circulating platelets, and/or platelets generated by the transfected megakaryocytes, in a subject in need thereof, wherein the method comprises administering to the subject a therapeutically effective amount of composition comprising naked RNA oligonucleotides.

### CROSS-REFERENCE TO RELATED APPLICATIONS

The present application claims priority under 35 U.S.C. § 119(e) to U.S. Provisional Application Nos. 63/391,002, filed Jul. 21, 2022; 63/439,365 filed Jan. 17, 2023; and 63/522,839, filed Jun. 23, 2023, all of which are hereby incorporated by reference herein in their entireties.

### STATEMENT REGARDING FEDERALLY SPONSORED RESEARCH OR DEVELOPMENT

This invention was made with government support under HL159006 awarded by National Institutes of Health. The government has certain rights in the invention.

### REFERENCE TO SEQUENCE LISTING

The Sequence Listing concurrently submitted herewith as a xml file named “205961-7088WO1(00343)_Sequence Listing.xml” created on Jul. 19, 2023 and having a size of 107.6 Kilobytes is herein incorporated by reference pursuant to 37 C.F.R. § 1.52(e)(5).

### BACKGROUND

Megakaryocytes (MKs) are large, nucleated cells, residing primarily in bone marrow and spleen where they develop from progenitor cells, and ultimately shed their extensive cytoplasm to generate blood platelets. Each mature megakaryocyte generates 1000-2000 platelets and releases them into the blood, at a rate of ˜10¹¹ new platelets generated per day in humans. The principal function of platelets is to plug vascular wounds. However, platelet plugs that grow too large can occlude (block) blood vessels (thrombosis), which remains a leading primary cause of morbidity and mortality worldwide. Platelet plugs also form and stabilize under a variety of conditions in the absence of vascular wounds, e.g., atherothrombosis. This action is the fundamental driver of adverse cardiac events including ischemic stroke, myocardial infarction (heart attack), generalized arterial and venous thrombosis, and venous thromboembolism including pulmonary embolism and deep vein thrombosis. Hence, the balance of platelet reactivity is essential to establish and maintain plug formation and prevent bleeding (hemostasis), without leading to thrombosis, and platelet hypo- and hyper-reactivity underlies dysfunction in hemostasis or thrombosis. Platelet production (thrombopoiesis) from megakaryocytes also plays directly and importantly into this balance: too many platelets (thrombocytosis) increase risk of thrombosis, whereas too few platelets (thrombocytopenia) increase risk of bleeding. Thrombosis is a major risk factor across many disease states, including cancers of nearly all types, as well as many if not most inflammatory states which together comprise abroad swath of morbid and deadly conditions.

Current standard-of-care for thrombosis includes both anticoagulant and antiplatelet approaches. The major antiplatelet drugs currently in wide use, though generally much improved in recent decades, still include risks of clinical bleeding that may be moderate to severe. Such severe bleeding includes intracranial hemorrhage. In addition, dysfunction in megakaryocyte development from stem and progenitor cells can lead not only to thrombocytosis or thrombocytopenia and the downstream clinical manifestations, but can also lead to myelodysplasias that may progress to hematologic malignancies. Thus, there are major gaps in clinical control of bleeding and clotting as well as control of blood cell development, providing an acute and long-term need for improved drugs and methods of treatment. The present invention addresses this unmet need.

### SUMMARY OF THE INVENTION

In one aspect, the invention provides a method of selectively modulating gene expression of one or more target genes in a cell of a subject, the method comprising administering to the subject a therapeutically effective amount of a composition comprising a naked RNA oligonucleotide, wherein the cell is at least one selected from the group consisting of a megakaryocyte, a circulating platelet, and a platelet generated from a megakaryocyte transfected with the naked RNA oligonucleotide.

In another aspect, the invention comprises a method of selectively modulating gene expression of one or more target genes in a cell, ex vivo or in vitro, the method comprising transfecting the cell with a naked RNA oligonucleotide.

In yet another aspect, the invention provides a method for selectively delivering a naked RNA oligonucleotide to a target tissue and/or target a cell of a subject, wherein the method comprises using the platelets transfected with the naked RNA oligonucleotide as a vehicle to deliver the naked RNA oligonucleotide to the target tissue and/or to the target cell. In certain embodiments, the target cell is a tumor cell, a leukocyte or an inflammatory cell. In certain embodiments, the target tissue comprises an endothelial cell.

In yet another aspect, the invention provides a composition for selectively modulating gene expression of one or more target genes in a cell of a subject, wherein the composition is a cell transfected with a naked RNA oligonucleotide.

In yet another aspect, the invention provides a composition for selectively modulating gene expression of one or more target genes in a cell, ex vivo or in vitro, wherein the composition comprises a naked RNA oligonucleotide within the cell.

In yet another aspect, the invention provides a kit comprising a composition having a naked RNA oligonucleotide for selectively modulating expression of at least one gene in a cell and an instructional material for use thereof.

In certain embodiments, the cell is at least one selected from the group consisting of a megakaryocyte, a platelet, and a platelet generated from a megakaryocyte transfected with the naked RNA oligonucleotide.

In certain embodiments, the composition comprises a cell transfected with a naked RNA oligonucleotide, wherein the cell is at least one selected from the group consisting of a megakaryocyte and a platelet.

In certain embodiments, the naked RNA oligonucleotide is selected from the group consisting of an miRNA, an siRNA, and any combination thereof.

In certain embodiments, the naked RNA oligonucleotide comprises a guide strand and a passenger.

In certain embodiments, the naked RNA oligonucleotide comprises a guide strand having a sequence selected from the guide strand sequences shown in Table 1.

In certain embodiments, the naked RNA oligonucleotide comprises a passenger strand having a sequence selected from the passenger strand sequences shown in Table 1.

In certain embodiments, the naked RNA oligonucleotide comprises at least one modification selected from the group consisting of a locked nucleic acid (LNA), a 2′-Fluorine base, and a 2′-O-methylated base.

In certain embodiments, the naked RNA oligonucleotide is thermostable. In certain embodiments, the naked RNA oligonucleotide is non-immunogenic. In certain embodiments, the naked RNA oligonucleotide is resistant to cleavage by riboendonucleases (RNAases).

In certain embodiments, the composition is administered intravenously.

In certain embodiments, the administering alters megakaryocyte development, platelet production, megakaryocyte function, and/or platelet function.

In certain embodiments, the subject is in need of:
i. an antiplatelet therapy,
ii. an anti-inflammatory therapy for treating thromboinflammation,
iii. a treatment for thrombocytopenia, thrombocytosis, and other pathological manifestations of disrupted megakaryocyte development,
iv. a platelet transfusion with platelets protected from platelet storage lesion,
v. a treatment for thrombosis,
vi. a treatment for acquired bleeding disorders, and/or
vii. a treatment for inherited bleeding disorders.

In certain embodiments, the composition comprises a saline solution.

In certain embodiments, the subject is a mammal. In certain embodiments, the mammal is a human.

In certain embodiments, the cell is derived from at least one selected from the group consisting of a cell culture system, a concentrate, a cell suspension, a tissue homogenate, an organoid, a tissue, and an organ.

In certain embodiments, the cell culture is a stem cell culture.

In certain embodiments, the concentrate is a platelet storage concentrate containing autologous plasma.

In certain embodiments, the transfection is performed without use of a synthetic carrier or an adjuvant.

In certain embodiments, the naked RNA oligonucleotide is suspended in an aqueous sterile saline solution.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1C show that naked double-stranded miRNA mimics selectively transfect bone marrow megakaryocytes in vitro. (FIG. 1A) Whole unfixed bone marrow cell suspensions captured on poly-L lysine (PLL)-coated slides were imaged live for AF488. Only megakaryocytes (MK) show green fluorescence, indicating MK-specific uptake. (FIG. 1B) Fixed bone marrow cells were counterstained for MK/platelet marker Cd41 and DAPI for nuclei. Only MKs, but not other bone marrow cells, show miLNA uptake. In this experiment, bone marrow cells were co-incubated with both the miLNA-AF488, and an unmodified version of miR-223-3p conjugated to AlexaFluor647. Lower panels show no fluorescence indicating degradation of the unmodified miRNA mimic. (FIG. 1C) Cells as in (FIG. 1B) counterstained for two MK/platelet markers Cd42d and Cd41, showing uptake by MKs at different stages including immature growth stages (small MK).

FIGS. 2A-2F show that infused naked double-stranded miRNA mimics selectively transfect bone marrow megakaryocytes and platelets in vivo. (FIG. 2A) Whole bone marrow cell suspensions extracted at 2 hrs after infusion, captured on poly-L-lysine (PLL)-coated slides and imaged live for AF488. Only megakaryocytes (MK) show green fluorescence, indicating MK-specific in vivo uptake. (FIG. 2B) Fixed bone marrow cells were counterstained for MK/platelet markers Cd42d and Cd41, and DAPI for nuclei. Only MKs and platelets, but not other bone marrow cells, show miLNA uptake, at 18 hours and 72 hours post-infusion. (FIG. 2C) Cells as in (FIG. 2B) analyzed by flow cytometry for miL uptake (by fluorescence), using Cd41/Cd42d and size (forward scatter FSC) to identify MKs. In this experiment spleens were also extracted and splenocyte suspensions were analyzed at 72 hours, together showing robust MK uptake and retention of the miLNA in bone marrow and splenic MKs. (FIG. 2D) Peripheral blood cells from the same animals as above were fixed and captured on coverslips, and platelets were identified with Cd42d antibodies. Platelet miLNA in vivo uptake efficiency approached 100%, as previously observed with washed in vitro platelets. Bar, 50 μm. (FIG. 2E) Composite high-mag image of platelets from (FIG. 2D). Bar, 10 m. (FIG. 2F) Blood cells and lung endothelium 2 hrs post-infusion. Rapid miLNAuptake in platelets but not WBCs, RBCs or endothelium (ECs).

FIGS. 3A-3D show that infused naked RhoA siLNAs suppress RhoA protein expression selectively in bone marrow megakaryocytes and modulate megakaryocyte development and platelet production in vivo. (FIG. 3A) RhoA siLNAs suppress RhoA protein expression selectively in bone marrow MKs after infusion. RhoA levels were measured by flow cytometry in permeablized whole bone marrow cell suspensions using RhoA antibodies, coupled with Cd42d antibodies to label MKs, and propidium iodide (PI) to label nuclear DNA. Left panel, RhoA expression levels in MKs as a function of ploidy class determined by PI staining. RhoA expression increased in larger, more mature MKs as expected due to expanded cytoplasm, but this increase was suppressed by RhoA siLNAs. Right panel, nucleated (PI+) non-MK bone marrow cells showed no change in RhoA protein levels. (FIG. 3B). RhoA siLNAs modulate RhoA-dependent MK function. RhoA knockout mice and RhoA-inhibitor-treated mice demonstrated that acute RhoA suppression leads to increased MK ploidy. RhoA siLNAs caused increased MK ploidy compared to control siLNA, demonstrating specific functional effects of RhoA suppression in MKs in vivo. (FIG. 3C) Increased peripheral blood platelet count with RhoA siLNA, corresponding to increased MK ploidy (FIG. 3B). (FIG. 3D) No change to mean platelet volumes with RhoA siLNA, confirming that increased MK ploidy drives higher platelet count but no generation of larger platelets.

FIGS. 4A-4B show that infused naked P2y12 (P2ry12) siLNA reduces platelet ADP activation response and prevents injury induced arterial thrombosis in WT mice, phenocopying current frontline antiplatelet drugs targeting P2Y12. (FIG. 4A) P2ry12 siLNA infusion reprograms platelets with reduced ADP reactivity. Platelet ADP response was tested by FACS in whole blood cell suspensions 24 hours after P2ry12 (open circles) or control scrambled (CTL, closed circles) siLNA infusion, for ADP-induced GpIIb/IIIa integrin activation with JonA antibodies (upper panel) and alpha-granule secretion with P-selectin antibodies (lower panel). p<0.05. n=3. (FIG. 4B) At 96 hours (day 5), anesthetized mice were subjected to arterial injury to the carotid artery by application of filter paper soaked in a solution of 7.5% FeCl₃ for 90 seconds (“Injury”). Blood flow was monitored with a Doppler probe downstream of the injury site for up to 30 minutes after injury. Arteries occluded, blocking blood flow, in control mice between 5-12 minutes after injury, as expected. In contrast, arteries in mice that had received naked P2ry12 siLNA (targeting the P2y12 ADP receptor, the target of current frontline antiplatelet antithrombotic drugs) remained patent and no occlusion was observed over the experimental time frame. N=3 each.

FIGS. 5A-5D show that single injection of naked miL-223-3p miRNA mimic transiently and potently increases platelet ADP reactivity and thrombotic response. (FIG. 5A) miL-223-3p injection led to a 4-fold mean increase in platelet ADP reactivity assessed 24 hours post-injection, as measured by flow cytometry for Jon/A antibodies recognizing the activated form of integrin GpIIb/IIIa, whereas reactivity was unaffected by control Cel-miL-67 or saline infusion. The increased reactivity was transient, indicating consumption of the targeting miLNA and reconstitution of the targeted proteins at later times. (FIG. 5B) The entire population of circulating platelets showed a rightward shift in ADP reactivity. This result suggests potent direct effects on circulating platelets, but effects on MKs cannot be ruled out. (FIG. 5C) Effects of miL-223-3p infusion on injury-induced arterial thrombosis was tested by FeCl₃ injury at 24 hours post-injection, as in FIG. 4B. miL-223-3p infusion potentiated the thrombotic phenotype. Shown are occlusion times in the carotid arteries after injury and removal of the FeCl3 insult. n=9 controls, n=6 miL-223-3p-treated mice. miL-223-3p infusion potentiated the thrombotic phenotype, demonstrating direct and potent effects of naked miLNAs on platelet physiological response. (FIG. 5D) Summary data for time to occlusion in the carotid artery, from FIG. 5C.

FIGS. 6A-6B show that a single or repeat addition of naked ADAM17 siRNA to platelets in storage concentrates protects against loss of GP1bα. (FIG. 6A) A single addition of naked ADAM17 siRNA directly to the platelet storage concentrates consistently maintained GP1bα total and surface expression in stored platelets for up to 48 hours. GP1bα levels after 48 hours dropped to similarly low levels as with control siRNA, indicating turnover of ADAM17 and GP1bα. (FIG. 6B) Multiple additions of ADAM17 siRNA to the stored platelet concentrates (by precipitating and rinsing the platelets, repeating transfection, and then reconstituting with reserved plasma), given at 0 and again at 48 hours, provided prolonged maintenance of GP1bα in the stored platelets.

FIG. 7A-7B show that a single addition of naked ADAM17 siLNA to platelet storage concentrates protect against loss of GP1bα function. (FIG. 7A) Shown is percent platelet agglutination (aggregation) over time following addition of ristocetein (Rs) at the indicated concentrations. A single addition of naked ADAM17 siLNA directly to the platelet storage concentrates on day 0, maintained GP1bα functional reactivity over the 5-day experimental time frame. These data provide the first demonstration of improvement of cellular physiological function in stored platelets by this approach. (FIG. 7B) GP1bα expression in the stored platelets.

FIG. 8 shows platelet-specific miLNA uptake: Platelet and platelet extracellular vesicle aggregates with leukocytes in peripheral blood, e.g., no uptake by leukocytes detected.

FIG. 9 shows normal CBC and megakaryocyte size (perploidy class) with RhoA siLNAs.

FIG. 10 shows normal CBC with miL-223-3p.

FIG. 11 shows normal CBC with P2ry12 siLNA.

FIG. 12 is an illustration outlining general approach in accordance with the embodiments of the invention: intravenous injection modeling infusion of modified small RNAs to selectively transfect megakaryocytes, as shown here, as well as circulating blood platelets.

FIG. 13 shows si/miLNA sequences designed and used for the experiments presented herein; some experiments used miR-223-3p mimic without the fluorophore tag. Sequences are shown 5′→3′ for both guide and passenger strands, which were hybridized before use to yield double-stranded oligonucleotides with overhangs at each terminus.

FIG. 14A-14C show miLNA uptake in MEG-01 cells only upon megakaryocytic differentiation. MEG-01 pre-megakaryocytic cells were cultured +/−PMA for different days as shown to induce megakaryocytic differentiation, then transfected in vitro for 1 hr with naked miL: AF488. (FIG. 14A) miLNA fluorescence, and percent miLNA-positive (miL+) cells are shown. miLNA uptake only appeared beginning at day 4 of differentiation. (FIG. 14B) CD41 expression onset (megakaryocyte/platelet marker) aligns with induced miLNA uptake ability on day 4. (FIG. 14C) miLNA uptake apparent in CD41+ cells.

FIG. 15 shows assessment of therapeutic window (TW) for P2ry12 siLNA and ticagrelor in mice. The TW was assessed as the separation between antithrombotic and bleeding effects, across a 3-log dose range of either ticagrelor or P2ry12 siLNA following intravenous administration. Antithrombotic effects were also tested in mice treated with a scrambled control siLNA at the highest dose tested for P2ry12 siLNA (500 μg/kg; n=5); results are shown in the large black square. The control siLNA had no protective effect against thrombosis, whereas P2ry12 siLNA had a dose-dependent protective effect against thrombosis. Thrombosis—squares and dashed line; inhibition of hemostasis—circles, and dotted lines. n=3−10.

FIG. 16 shows representative examples from FeCl₃ arterial injury assays for occlusive thrombosis.

FIG. 17 shows representative Doppler blood flow tracings after carotid arterial injury, 24 hrs after IV administration of P2ry12 siLNA. Right panel shows occlusion stability as a function of P2y12 suppression measured by FACS. Lower left (FIG. 17 continued) shows occlusion time as a function of siLNA IV dose. The experiment is stopped at 20 min (1200 seconds); any measure of 1200 s indicates restored blood flow at the end of the experimental time frame.

FIG. 18 shows diversity in reactivity amongst the circulating platelet population.

FIG. 19 shows platelet hPAR4 suppression by F2RL3 siLNA in hPAR4 mice. Platelet lysates were extracted 24 hours after single injection of F2RL3 (hPAR4) siLNA into the hPAR4 mice, and immunoblotted with PAR4 antibodies. hPAR4 TG/mPar4 KO indicates mice transgenic for human PAR4, and deleted for the mouse Par4 gene.

FIG. 20 shows that F2RL3 siLNA reprograms platelets with reduced reactivity to PAR4 agonist. hPAR4 mice were infused with 0.5 mg/kg F2RL3 or control siLNA, and platelet PAR4-activating peptide (PAR4-AP) response was tested in platelets from whole blood cell suspensions with Jon/A antibodies indicating integrin activation (left) and P-selectin (right) antibodies indicating granule secretion, by flow cytometry after 24 hours. n=6.

FIG. 21 shows that F2RL3 siLNA provides protection from thrombosis with minimal effects on bleeding. hPAR4 TG/mPar4 KO mice were injected with 0.5 mg/kg F2RL3 or control siLNA, and subject to FeCl3 thrombosis (left) or tail tip amputation bleeding (right) at 24 hrs. Bleeding is shown as a ratio of blood weight loss (g) to starting mouse weight (g). n=3.

FIG. 22A-D illustrates in vivo siLNA-mediated silencing of Gsα (mRNA, Gnas) in murine platelets at 24 hrs, pro-hemostatic effects of Gnas and Ptgir siLNAs, and rescue of acquired (ticagrelor-induced) bleeding with Gnas and Ptgir siLNAs. FIG. 22A shows Gsα suppression by Gnas siLNA, in murine platelets at 24 hrs after tail vein injection of siLNA. n=3. FIG. 22B shows reduced bleeding, as measured in FIG. 21, in mice treated with Gnas (Gsα) or Ptgir (IP, prostacyclin receptor) siLNAs. N are shown for each. FIG. 22C shows accelerated hemostasis in mice with Gnas siLNA compared to controls, after 1.5-mm diameter tail tip amputation. The experiment was stopped at 20 minutes (1800 seconds). N=6. FIG. 22D shows rescue of acquired bleeding induced by 0.5 mg/kg ticagrelor, in mice treated with Gnas and Ptgir combined siLNAs at 24 hrs after siLNA injection. Ticagrelor or vehicle was given 5 minutes before the start of the bleeding assay. *, p<0.04. n=3−10.

FIG. 23 show dynamic Gsα expression over the platelet lifespan. Gsα expression ratios in new (dotted lines) and extant (solid line) platelets to total platelets are shown ±s.e.m. Gsα was monitored by flow cytometry using Gsα-specific antibodies in fixed, permeablized platelets, extracted at the times indicated after pulse labeling with Gp1bβ antibodies. n=3.

FIGS. 24A-24B show selective in vivo miLNA uptake by (FIG. 24A) blood platelets and (FIG. 24B) bone marrow megakaryocytes. Cells were collected from un-infused WT and miR-223 KO mice (−), and miR-223 KO mice 18 hrs after miL-223-3p infusion (+), labeled with surface markers, and single-cell FACSorted for each population as indicated. The presence of miR-223-3p was detected in each population by PCR from poly(dA)-tailed cDNA as in prior studies, as direct demonstration of cell type-specific in vivo miLNA uptake in platelets/MK.

FIG. 25 shows that anti-miRs/antagomiRs do not harbor the unique property of platelet-/megakaryocyte-specific internalization and utilization that miLNAs and siLNAs do. An 8-nucleotide antagomiR (anti-miR) comprised of all locked nucleic acids, with a 3′-FAM fluorophore was injected into mice, and FAM fluorescence of the indicated cell types was monitored by flow cytometry.

FIG. 26 shows targeted effects of siLNAs by subdermal administration using osmotic pumps in mice. 3-day-release pumps harboring 0.6 mg/kg P2ry12 (red open circles) or control (blue closed circles) siLNA were implanted subdermally in mice, and platelet ADP-induced integrin activation (left) and P-selectin exposure (right) were assessed daily by flow cytometry. Data are shown as fold area under curve for ADP dose ranges as in FIG. 4, ±s.e.m. n=3. *, p<0.03; **, p<0.05.

### DETAILED DESCRIPTION OF THE DISCLOSURE

In the present invention, the overall approach is to introduce microRNA mimics (miRNAs) and/or short interfering RNAs (siRNAs) into megakaryocytes and/or platelets, to modulate expression of their cognate target genes, which contribute to the specific cellular, physiological, and pathophysiological functions as outlined above. mi/siRNAs suppress translation of their target mRNAs in a sequence-specific manner to prevent expression of the cognate proteins. Thus, mi/siRNAs suppress protein expression of target genes.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

The term “alteration” or “alter” or “modulate” refers to a change (increase or decrease) in the expression levels or activity of a gene or polypeptide as detected by standard art known methods such as those described herein. In some embodiments, an alteration in expression level includes a 10% change in expression levels, a 25% change, a 40% change, and a 50% or greater change in expression levels.

As used herein, the term “antimiR” refers to chemically modified, single-stranded antisense oligonucleotides that inhibit miRNA function. The length of antimiRs ranges from seed-targeting 8-mer oligonucleotides to antimiRs that are fully complementary to mature miRNAs. As synthetic reverse complements, they prevent miRNA activity by competing with the target 3′UTR mRNA site for miRNA binding.

As used herein, the term “antagomiR” refers to 3′ cholesterol-conjugated, 2′-O-methyl-modified antisense oligonucleotides that inhibit miRNA function. The antagomiRs are fully complementary to mature miRNAs.

As used herein, the term cell refers to a cell (such as, for example, a megakaryocyte) and/or to a cell fragment (such as, for example, a platelet).

A disease or disorder is “alleviated” if the severity of a symptom of the disease or disorder, the frequency with which such a symptom is experienced by a patient, or both, is reduced.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. RNA cleavage can result in the production of either blunt ends or staggered ends.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “expression” as used herein is defined as the transcription and/or translation of a particular nucleotide sequence driven by its promoter.

As used herein, the term “modulating gene” refers to modulating gene and/or gene products and/or any genetic material. In certain embodiments “modulating gene” refers to, for example, modulating an mRNA, a pre-mRNA, a lncRNA, a snRNA, a snoRNA, a tRNAs, a rRNAs, a YRNAs, piRNAs.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “locked nucleic acid (LNA)” as used herein refers to modified RNA nucleotide in which the ribose moiety is modified with an extra bridge connecting the 2′ oxygen and 4′ carbon.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, the term “newly generated platelets” refers to the platelets produced by megakaryocytes between 0 and 48 hours relative to siLNA or miLNA in vivo administration or ex vivo or in vitro transfection.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein the term “naked RNA oligonucleotides” refers to RNA oligonucleotides without encapsulation in liposomes or nanoparticles of any kind, or anchored to any other molecules or adjuvants. The naked RNA oligonucleotides include, for example, microRNA mimics (miRNAs) or short interfering RNAs (siRNAs).

“Parenteral” administration of an immunogenic composition includes, e.g., subcutaneous (s.c.), intravenous (i.v.), intramuscular (i.m.), or intrasternal injection, or infusion techniques.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, “polynucleotide” includes cDNA, RNA, DNA/RNA hybrid, antisense RNA, siRNA, miRNA, snoRNA, tRNA, YRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences. As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

By “small interfering RNA” or “siRNA” is meant a double stranded RNA. Optimally, an siRNA is 18, 19, 20, 21, 22, 23 or 24 nucleotides in length and has base overhang at its 3′ end. These dsRNAs can be introduced to an individual cell or to a whole animal; for example, they may be introduced systemically via the bloodstream. Such siRNAs are used to downregulate mRNA levels or promoter activity.

The terms “siLNA” and “miLNA” refer to LNA-modified siRNA molecules and LNA modified miRNA molecules, respectively.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

A “target site” or “target sequence” refers to a genomic nucleic acid sequence that defines a portion of a nucleic acid to which a binding molecule may specifically bind under conditions sufficient for binding to occur.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The purpose of this invention is to provide a platform for modulating megakaryocyte development, platelet production, and/or platelet reactivity in vivo, ex vivo and in vitro to improve all of the clinical scenarios disclosed elsewhere herein, and to prolong stored platelet life and function with reduced inflammatory response in stored platelet units.

Without wishing to be limited by any theory, the cellular physiological basis of the invention is the finding that megakaryocytes and platelets have the unique ability to internalize naked, unencapsulated small RNAs, due to the unique membrane structure of these cells. This feature provides the direct ability to transfect megakaryocytes and platelets with modulatory small RNAs, with minimal off target tissue transfection, in vivo, ex vivo, and in vitro.

Importantly, although unassisted uptake (also known as ‘gymnosis’) of oligonucleotides has been explored for therapeutic use, the uptake efficiency and tissue targeting remain unsolved problems, which are solved by this invention for megakaryocytes and platelets.

### Compositions

In one aspect, the invention provides a composition for selectively modulating gene expression in megakaryocytes (MKs) and/or platelets and/or platelets generated by the transfected megakaryocytes. The composition comprises a naked, e.g., an unencapsulated, RNA oligonucleotide. In certain embodiments, the RNA oligonucleotide is suspended in an aqueous solution but without encapsulation in liposomes or nanoparticles of any kind or without anchoring to any other molecules or adjuvants In certain embodiments, the aqueous solution is, for example, a sterile saline solution.

In certain embodiments, the RNA oligonucleotide is selected from the group consisting of a double-stranded (ds) miRNA mimic containing modified RNA base(s), in which the guide strand represents the nucleotide sequence of a native miRNA, an siRNA, and any combination thereof. In certain embodiments, the RNA oligonucleotide can be synthesized easily and cheaply.

In certain embodiments, the RNA oligonucleotide selectively transfects megakaryocytes and platelets in vivo, ex vivo, or in vitro. In certain embodiments, the in vivo transfection is used, for example, to modulate megakaryocyte development, function and gene expression, platelet production, and platelet in vivo functions including, control of bleeding, thrombosis, and inflammation. In certain embodiments, the ex vivo transfection is used, for example, for improved functional lifespan and/or reduced inflammatory state of stored platelet concentrates. In certain embodiments, the in vitro transfection is used, for example, for stem cell therapeutic approaches such as modulation of megakaryocytes derived from induced pluripotent stem cells.

In certain embodiments, the RNA oligonucleotide is designed to avoid degradation by plasma and tissue riboendonucleases (RNAses). In certain embodiments, the RNA oligonucleotide is non-immunogenic. In certain embodiments, the RNA oligonucleotide is designed to avoid or to provoke little or no immune response.

In certain other embodiments, the RNA oligonucleotide optionally comprises modified nucleotide(s) such as, for example, locked nucleic acid(s) (LNA(s)) which is/are modified to include a methylene bridge bond between the 2′ oxygen and the 4′ carbon of the pentose ring and 2′-OMe RNA base(s), which are methylated at the 2′-oxygen and/or 2′-Fluorine (2′-F) base(s).

In certain embodiments, for the RNA nucleotide, wherein the 3′ terminal residues in the guide strand overhang include Urasils(Us), U(s) is/are replaced with Thymine(Ts) (sinceUracil cannot be modified as an LNA due to its structure) as an LNA (T).

The LNA-based RNA oligonucleotides are known to confer strong resistance to RNA cleavage by RNAses, which are enriched in all tissues including blood/plasma, bone marrow, spleen, and other hematopoietic niches, which are the target tissues for the RNA oligonucleotides of the present invention. In addition, the precise placement of LNA and 2′-OMe RNA nucleotides within the double-stranded miRNA mimic, the siRNA, or the antmiR, confer several critical properties; these include for the guide strand: a 3′ overhang consisting of 2 or 3 LNA bases to favor guide strand incorporation into the RNA-induced silencing complex (RISC) but avoiding loss of suppressive activity; for the passenger strand: truncated 5′ end plus a 5′ terminal LNA base to inactivate the strand, moderate LNA bases (5-6) distributed evenly in the strand to confer RNase resistance, diuridine at the 3′ end to provide an overhang and further render the strand non-functional, 2′-OMe modifications of the 3′-end uridine and most or all adenosines to greatly reduce immunogenicity. miRNA mimic guide strand sequences are determined by the native sequence of the given mature miRNA.

In certain embodiments, in place of 2′-OMe nucleotides, 2′-F nucleotides would also suffice for those bases in the passenger strand of the composite double stranded si or miLNA that would be substituted with 2′-OMe, to yield similar effects: protection from riboendonucleases, and avoidance of inducing an immune response.

In certain embodiments the siRNA is about 15 to about 30 nucleotides in length and is designed to target an mRNA of interest. In certain embodiments, the siRNA is about 15, 16, 17, 18, 19, 20, 21, 22, 23, 24, 25, 26, 27, 28, 29, or about 30, nucleotides long.

In certain embodiments, the antmiRs comprises the reverse complement sequence of the 5′ end of the target miRNA, typically nucleotides 1-8 but not limited to that region. In certain embodiments, the antmiRs are comprised entirely of LNA bases.

In certain embodiments, the RNA oligonucleotide comprises two strands, a guide strand and a passenger strand. In certain embodiments, the guide strand sequences and the passenger strand sequences are as listed in Table 1. In certain embodiments, the guide strand has a sequence that is substantially identical to a guide strand sequence shown in Table 1. In certain embodiments, the passenger strand has a sequence that is substantially identical to a passenger strand sequence shown in Table 1. In certain embodiments, the guide strand has a sequence that is at least 80%, 85%, 90%, 95%, 96%, 97%, 98%, or 99% identical to a guide strand sequence shown in Table 1. In certain embodiments, the passenger strand has a sequence that is at least 80%, 85%, 90%, 95%, 96%, 97%, 98%, or 99% identical to a passenger strand sequence shown in Table 1.

In certain embodiments, the naked RNA oligonucleotide comprises only unmodified nucleotides.

In certain embodiments, the RNA oligonucleotide is thermostable i.e. stable at frozen/deep freeze temperatures, refrigerated temperatures, room temperature and body temperature.

In certain embodiments, the composition is administered to a subject to enter their blood circulation. In certain embodiments, the subject is a mammal. In certain embodiments, the subject is a human subject.

In another aspect, the invention provides a composition for selectively modulating gene expression of one or more target genes in a cell of a subject, wherein the composition comprises a cell transfected with a naked RNA oligonucleotide, wherein the cell is at least one selected from the group consisting of a megakaryocyte, a circulating platelet, and a platelet generated from a megakaryocyte transfected with the naked RNA oligonucleotide.

In yet another aspect, the invention provides a composition for selectively modulating gene expression of one or more target genes in a cell, ex vivo or in vitro, wherein the composition comprises a cell transfected with a naked RNA oligonucleotide, wherein the cell is at least one selected from the group consisting of a megakaryocyte and a platelet.

In certain embodiments, the naked RNA oligonucleotide is as described elsewhere herein.

### Methods

In another aspect, the invention provides a method of selectively modulating gene expression of one or more target gene(s) in a cell of a subject, the method comprising administering to the subject a therapeutically effective amount of a composition comprising a naked RNA oligonucleotide. In certain embodiments, the cell is at least one selected from the group consisting of a megakaryocyte, a circulating platelet, and a platelet generated from a megakaryocyte transfected with the naked RNA oligonucleotide. In certain embodiments, the composition comprising the naked RNA oligonucleotide is as described elsewhere herein.

Although platelets do not contain nuclei, they do have other forms of genetic material that regulate protein expression, and modulating gene expression include interactions with downstream gene products such as, for example, mRNA.

In certain embodiments, the term “modulating gene” includes modulating a gene and/or a gene product and/or any genetic material. In certain embodiments “modulating gene” includes but is not limited to modulating an mRNA, a pre-mRNA, a lncRNA, a snRNA, a snoRNA, a tRNAs, a rRNAs, a YRNAs, piRNAs.

In certain embodiments, the composition is administered, for example, subcutaneously, intravenously, intramuscularly, or intraperitoneally. In certain embodiments, the composition is administered intravenously. In certain embodiments, the composition is administered topically.

In certain embodiments, the administrating results, for example, in altered megakaryocyte development, platelet production, megakaryocyte function, and/or platelet function.

In yet another aspect, the invention provides a method for an improved antiplatelet therapy over standard-of-care pharmacological agents, for antithrombotic uses with reduced risk of clinical bleeding. In certain embodiments, the method comprises administering to the subject in need thereof a therapeutically effective amount of the composition described elsewhere herein.

In certain embodiments, antiplatelet therapy target includes, but is not limited to, P2Y12 (ADP receptor, gene P2RY12). This protein product is the target of current frontline antiplatelets including cangrelor, ticagrelor, and clopidogrel (Plavix), used worldwide as antithrombotics but which incur variable risk of clinical bleeding that can range from moderate to severe, such as life-threatening intracranial hemorrhage. MK/platelet-specific knockdown of P2Y12 by this platform may provide improved antithrombotic utility with reduced bleeding risk compared with total pharmacological blockade, affecting many patients in many clinical scenarios of thrombosis risk reduction and management.

In certain embodiments, antiplatelet therapy target includes, but is not limited to, PAR1 (thrombin receptor). This protein is the target of vorapaxar, which was pulled from most clinical use because it causes bleeding. MK/platelet-specific knockdown of PAR1 by this platform may provide an alternative to vorapaxar with improved antithrombotic utility with reduced bleeding risk, affecting many patients in many clinical scenarios of thrombosis risk reduction and management.

In yet other embodiments, the antiplatelet therapy target includes, but is not limited to, FcγRIIa (gene, FCGR2A). This is the antibody receptor on platelets and other blood cells that mediates immunothrombosis, e.g., thrombosis in response to increased immune activity. MK/platelet-specific knockdown of FcγRIIa by this platform may provide strong protection from immunothrombosis across a variety of clinical settings of inflammation and for many patient cohorts, with improved specificity of action and therefore reduced side effects compared to current standard of care.

In yet other embodiments, the antiplatelet therapy targets include, but are not limited to, platelet intracellular proteins involved in reactivity. These include a long list of putative targets that, similar to those listed above, can be modified by this platform technology in a personalized medicine approach based on the individual patient's platelet expression profile. For example (one of many possible), patients with over-expressed cytoplasmic suppressors of platelet receptor function, associated with reduced platelet responsiveness secondary to bleeding diathesis, may be treated by targeting the over-expressed suppressor protein(s).

In yet another aspect, the invention provides a method for treating platelet-induced inflammation, thromboinflammation, wherein the method comprises administering to the subject in need thereof a therapeutically effective amount of the composition described elsewhere herein.

In certain embodiments, targets for treating thromboinflammation include, but are not limited to platelet-derived inflammatory cytokines and their upstream regulators. These include, but are not limited to, interleukins and their upstream inducers such as NLRP3, CCL lymphocyte activators, and others. Platelets contribute to inflammation by release of inflammatory cytokines; for example, contributing to hyperinflammatory response in sepsis which is a major cause of mortality. However, total blockade of inflammation in many scenarios compromises necessary patient immune response. MK/platelet-specific knockdown of inflammatory cytokines by this platform may provide improved anti-inflammatory utility with maintenance of necessary immune function.

In other embodiments, target for treating thromboinflammation includes, but is not limited to PAR4 (secondary thrombin receptor). PAR4 antagonists are also utilized as antiplatelet and anti-inflammatory drugs. PAR4 plays a major role in platelet generation of inflammatory material. However, total blockade of PAR4 in many scenarios may be undesirable. MK/platelet-specific knockdown of PAR4 by this platform may provide improved anti-inflammatory utility with maintenance of necessary immune function.

In yet other embodiments, targets for treating thromboinflammation include, but are not limited to intracellular platelet proteins (there are multiple targets in this category) that mediate generation of platelet-derived microvesicles, which is an active process driven by the actions of multiple proteins, that occurs as a result of platelet stimulation such as by vascular injury, trauma, as a result of thrombolytic or thrombectomy treatment of ischemia, or in atherosclerotic and atherothrombotic settings. These microvesicles are both proinflammatory and procoagulant. This platform technology will reduce generation of platelet microvesicles, and/or reduce expression of the proinflammatory or procoagulant components, to yield improved anti-inflammatory and anticoagulant outcomes over current standard of care.

In yet another aspect, the invention provides a treatment for acute bleeding and bleeding disorders, including acquired and inherited bleeding disorders. These treatments may be considered as antibleeding or pro-hemostatic therapies. Bleeding disorders treated with the invention include, but are not limited to, platelet reactivity disorders and coagulation disorders. Acquired bleeding disorders include, but are not limited to, those induced by antiplatelet or anticoagulant therapies. Targets for antibleeding/pro-hemostatic therapies include, but are not limited to, endogenous negative regulatory proteins expressed in platelets such as Gsα (gene name, GNAS), IP (prostacyclin receptor, gene name PTGIR), GSK3β, and other modulators of platelet reactivity.

In yet another aspect, the invention provides a treatment for thrombocytopenia, thrombocytosis, and other pathological manifestations of disrupted megakaryocyte development, wherein the method comprises administering to the subject in need thereof a therapeutically effective amount of the composition described elsewhere herein.

These conditions are driven by alterations in gene expression in developing megakaryocytes, such as upregulation of transcription factors that suppress MK development in favor of erythrocyte lineage leading to thrombocytopenia, or conversely, factors that drive platelet production leading to thrombocytosis. Targeting these upregulated mediators of altered cell fates may be used to restore normal MK development and normal platelet production.

In yet another aspect, the invention provides a method for improved treatment for platelet storage lesion, as outlined above. In certain embodiments, the method comprises administering to the subject in need thereof a therapeutically effective amount of the composition described elsewhere herein. In certain embodiments, the target for treating platelet storage lesion includes, but is not limited to, ADAM17 metalloproteinase responsible for cleavage of platelet vWF receptor GP1bα. In certain embodiments, the target for treating platelet storage lesion includes, but is not limited to, ADAM10 metalloproteinase, responsible for cleavage of platelet collagen receptor GPVI. In certain embodiments, the target for treating platelet storage lesion includes, but is not limited to, miR-326 (using antmiR-326) to de-repress anti-apoptotic master regulator BCL×L. In certain embodiments, the target for treating platelet storage lesion includes, but is not limited to, Neuraminidase I, which is responsible for desialylation of platelet surface proteins leading to accelerated clearance of stored platelets after transfusion. In certain embodiments, the targets for treating platelet storage lesion include, but are not limited to, inflammatory cytokines as described elsewhere herein, and proteins involved in microvesicles as describe elsewhere herein, each of which are released by platelets over time in storage leading to proinflammatory state of the platelet storage concentrates. In yet other embodiments, other mediators of platelet death, dysfunction, and inflammatory state in storage are also targets for this platform technology.

In yet another aspect, the invention provides improved treatment for thrombosis risk as relates to hormonal effects including gender disparities for risk of thrombosis, thrombosis risk increased with age and menopause, estrogen therapy and hormone replacement therapies, etc. In certain embodiments, the method comprises administering to the subject in need thereof a therapeutically effective amount of the composition described elsewhere herein. In certain embodiments, the target for treating thrombosis includes, but is not limited to, Estrogen Receptor beta (ER3, gene ESR2), which is the major estrogen receptor in MKs/platelets with established non-nuclear functions altering platelet reactivity and associated with thrombosis.

In yet other embodiments, the targets for treating thrombosis include, but are not limited to genes involved in hormonal response, such as adrenergic, androgen, and other hormone receptors which are expressed in platelets. In certain embodiments, targeting these genes reduce platelet reactivity without compromising hormone bodily functions, by specific targeting to MKs/platelets. Together this group of targets represents a major area for improvement over current standard of care.

In yet another aspect, the invention provides a method of selectively modulating gene expression of one or more target genes, in vitro or ex vivo, in megakaryocytes or platelets derived from at least one selected from a cell culture system, a concentrate, a cell suspension, a tissue homogenate, an organoid, a tissue, and an organ, wherein the method comprises transfecting the megakaryocytes or the platelets with the naked RNA oligonucleotide. In certain embodiments, the target gene or the target protein is at least one selected from those listed in Table 1.

In certain embodiments, the platelets (in vivo or in vitro) transfected with naked RNA oligonucleotides of the present invention can be used as a delivery vehicle to deliver those naked RNA oligonucleotides to other target cells and/or target tissues, in vivo, including for example, tumor cells, leukocytes, inflammatory cells, endothelial cells, etc. Delivery mechanisms from transfected platelets to heterologous cells and tissues may include extracellular vesicles released by the transfected platelets, which may include microparticles (also known as microvesicles), exosomes, ectosomes, or apoptotic bodies, or other platelet releasates harboring the transfected naked RNA oligonucleotides. In the example of tumor cells, the solid tumor cells due to their leaky vasculature allow for the platelet microparticles carrying the naked RNA oligonucleotide to reach the tumors. Hence, the mi/siRNA transfer from the platelets is likely to be selective for the tumor cells over the cells in normal tissues, which the platelet microparticles don't have an easy access to.

In certain embodiments, the invention provides a method of improved cell culture treatment for in vitro MKs such as isolated directly or derived from stem cell cultures, for bone marrow reconstitution and for industrial platelet production. In certain embodiments, the method comprises transfecting the MKs with the at least one RNA oligonucleotide described elsewhere herein.

In certain embodiments, the cell culture is, for example, a stem cell culture.

In certain embodiments, the concentrate is, for example, a platelet storage concentrate containing autologous plasma. In certain embodiments, the gene expression is modulated in platelets to prolong stored platelet life and function with reduced inflammatory response in stored platelet units.

In certain embodiments, transfection is achieved ex vivo by, for example, direct addition to platelet storage units containing autologous plasma, or to any cell suspension containing megakaryocytes and platelets (including but not limited to blood, bone marrow cells, splenocytes, lung or other tissue homogenates, cell suspensions, organoids, tissues, or organs), or to in vitro cultured megakaryocytes or platelets. The mi/siRNAs/antmiRs selectively transfect megakaryocytes and platelets and modulate their function as driven by changes in expression of the target gene(s).

In certain embodiments, the transfection is performed without use of synthetic carrier or adjuvants.

The composition and methods described herein are improvements over lentivirus or other approaches that carry risks associated with gene therapeutic approaches using active viruses, and by increasing platelet production.

In certain embodiments, the subject is a mammal. In certain embodiments, the subject is a human subject. In certain embodiments, the subject is in need of at least one selected from the group consisting of an antiplatelet therapy, an anti-inflammatory therapy for treating thromboinflammation, a treatment for thrombocytopenia, thrombocytosis, and other pathological manifestations of disrupted megakaryocyte development, a platelet transfusion with platelets protected from platelet storage lesion, a treatment for thrombosis, a treatment for acquired bleeding disorders, and a treatment for inherited bleeding disorders.

### Kit

In yet another aspect, the invention provides a kit comprising a composition including a naked RNA oligonucleotide for selectively modulating expression of at least one gene in megakaryocytes and/or platelets and an instructional material for use thereof. In certain embodiments, the composition is used for modulating gene expression in vivo and/or ex vivo and/or in vitro.

In yet another aspect, the invention provides a kit comprising a composition including a cell transfected with a naked RNA oligonucleotide for selectively modulating expression of at least one gene in megakaryocytes and/or platelets and an instructional material for use thereof. In certain embodiments, the composition is used for modulating gene expression in vivo and/or ex vivo and/or in vitro. In certain embodiments, the composition is as described elsewhere herein. In certain embodiments, the naked RNA oligonucleotide is as described elsewhere herein.

### Pharmaceutical Compositions

The compositions or pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Pharmaceutical compositions of the present invention may be administered in solid or liquid form such as tablets, capsules, powders, solutions, suspensions, emulsions and the like. Pharmaceutical compositions of the present invention may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes. In some embodiments, the composition may be applied to the nose, throat or bronchial tubes, for example by inhalation.

The dosage of the above treatments to be administered to a patient will vary with the precise nature of the condition being treated and the recipient of the treatment and can be determined by physical and physiological factors such as body weight, severity of condition, previous or concurrent therapeutic interventions, and on the route of administration. The scaling of dosages for human administration can be performed according to art-accepted practices. The dose for a miR mimic, for example, will generally be in the range 1 to about 100 mg for an adult patient, usually administered monthly for a period between 1 and 12 months. The preferred monthly dose is 1 to 10 mg per month although in some instances larger doses of over 10 mg per month may be used.

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 1 microgram/kg/body weight, from 5 microgram/kg/body weight, 10 microgram/kg/body weight, 50 microgram/kg/body weight, 100 microgram/kg/body weight, 200 microgram/kg/body weight, 350 microgram/kg/body weight, 500 microgram/kg/body weight, 1 milligram/kg/body weight, 5 milligram/kg/body weight, 10 milligram/kg/body weight, 50 milligram/kg/body weight, 100 milligram/kg/body weight, 200 milligram/kg/body weight, 350 milligram/kg/body weight, or 500 milligram/kg/body weight, to 1000 mg/kg/body weight or more per administration, and any range derivable therein. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 1 micrograms compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular subject. Those of skill in the art would recognize the conditions and situations warranting modified dosing.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventor regard as his invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### Experimental Examples

The invention is further described in detail by reference to the following experimental examples. These examples are provided for purposes of illustration only and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods

Double-Stranded RNA Design and Synthesis and ‘Naked’ dsRNA Preparation.

About 19-29 nucleotide (nt) guide and passenger strand RNAs for double-stranded siRNA duplexes were designed from the full target mature (spliced) mRNA sequences using siDesign (sidirect2.rnai.jp/) following the parameters outlined in Amarzguioui. M et al, Biochem Biophys Res Commun 316, 1050-1058, (2004). PMID: 15044091; Reynolds, A. et al. Nature biotechnology 22, 326-330, (2004). PMID: 14758366; Ui-Tei, K. et al. Nucleic Acids Res 32, 936-948, (2004). PMID: 14769950; PMCID: PMC373388, and prime candidates are selected from the highest scoring candidates using the scoring method in Fakhr, E. et al. Cancer gene therapy 23, 73-82, (2016). PMID: 26987292, with a minimum score of 14. The candidate sequences were designed by the software to incorporate 2-nt overhangs at each 3′ end of the duplex. Double-stranded miRNA mimics were designed as the full-length sequence of the mature miRNA as the guide RNA strand, and the passenger strand is the exact reverse complement sequence, truncated in the first 2 or 3 5′-nts to generate a 3′ overhang in the guide strand, and with diuridine (UU) appended to the 3′ end to yield a UU overhang in the duplex. Dithymidine (TT) can also be substituted in place of UU. The sequences were then modified by the following rules: 1) the 2-nt 3′ overhang sequence in the passenger strand for siRNAs was replaced with diuridine (UU) (for miRNA mimics, a UU was added to the passenger strand sequence as described above); 2) the 2 or 3-nt 3′ overhang nts in the guide strand were converted to corresponding locked nucleic acids (LNA) (note that the 3′ terminal nt in the guide strand must be converted to the corresponding LNA but the others do not have to be, though it is preferable. Additionally U residues generally cannot be made into LNA therefore, wherever a U appears in the overhang, it wouldn't be LNA. Further, the nt added at the 3′ terminal can never be U because design rules for sequence selection enforce either G or C at the terminal position); 3) the first 5′-terminal nt in the passenger strand was converted to the corresponding LNA; 4) adenosines (A) in the passenger strand were converted to corresponding 2′-oxymethyl (2′-OMe) A nts, at a minimum of five 2′-OMe A nts, and distributed as evenly as possible across the passenger strand sequence. If A nts are limited, U nts were converted to 2′-OMe U nts to yield a total of at least five 2′-OMe A or U nts; 5) the second U in the diuridine (UU) overhang at the 3′ terminus of the passenger strand was also converted to a 2′-OMe U; 6) in addition to the 5′-terminal LNA nt in the passenger strand (which cannot be U), five C, G or A nts in the passenger strand were converted to corresponding LNA nts, distributed as evenly as possible across the passenger strand sequence. The siRNAs and miRNA mimics with the above modifications were synthesized and annealed as duplexes, purified, and validated, by standard oligonucleotide synthesis. Purified RNA duplexes were suspended in buffer made from sterile deoxy/deionized water, aliquoted and stored at −20° C. or −80° C. for up to one year. Freeze/thaw cycles are not recommended but are not likely to reduce efficiency substantially if kept to a minimum. RNA duplexes can be combined by mixing the suspensions, for multiplex treatments as needed for the specific indication.

### In Vivo Transfection.

The naked dsRNA duplexes in sterile liquid suspension were administered at empirically determined concentrations to the blood circulation in the subject. Administration can be through any route to reach the peripheral blood circulation, including but not limited to intravenous or intraarterial via needle, catheter or any other direct administration route to the blood vasculature, or through indirect routes, e.g., intramuscular, subdermal, transdermal application directly or absorbed into a patch, intraocular, intranasal, or intraperitoneal. Oral administration may be feasible. The dosage and frequency of administration are dependent on the target gene and specific indication. Typically, naked dsRNAs were given at approximately 0.2 mg/kg daily, but this may vary. The overall duration of administration also varies by indication.

### Ex Vivo and In Vitro Transfection.

The naked dsRNA duplexes in sterile liquid suspension were added directly to stored platelet concentrates or in vitro cultured megakaryocytes or platelets, at empirically determined concentrations for each desired target mRNA or mRNAs. Typically, the concentration is between about 100-1000 nM. Transfection typically begins at or near the time of collection of platelet concentrates from donors, or megakaryocyte or platelet culture, but may be started at any time based on the indication and may be repeated as frequently as once per day throughout the duration of storage prior to transfusion, or cell culture, as needed depending on the indication.

TABLE 1
Shows target genes and the RNA oligonucleotide sequences for targeting those genes.

| siLNA/ | | | | | | | Guide | Passenger | Guide | Passenger |
|---|---|---|---|---|---|---|---|---|---|---|
| miLNA | Target | | | | | | strand | strand | strand | strand |
| or | gene | | Appli- | Appli- | Indi- | Indi- | sequence | sequence | sequence | sequence |
| antago | name | Target | cation | cation | cation | cation | #1 | #1 | #2 | #2 |
| miR | (mRNA) | protein | 1 | 2 | 1 | 2 | 5′-->3′ | 5′-->3′ | 5′-->3′ | 5′-->3′ |
| siLNA | P2RY12 | P2Y12 | In | | Anti- | Anti- | AGAGU | GCAUU | AGAA | CUGAU |
| | | | vivo | | throm- | throm- | AAGAA | CAUGU | UCAU | CUUCU |
| | | | | | botic - | botic - | CAUGA | UCUUA | GAGA | CAUGA |
| | | | | | chronic | acute | AUGCC | CUCUU | AGAU | UUCUU |
| | | | | | | | C | U(SEQ | CAGA | U(SEQ |
| | | | | | | | (SEQ | ID | A | ID |
| | | | | | | | ID | NO: | (SEQ | NO: |
| | | | | | | | NO: | 33) | ID | 91) |
| | | | | | | | 1) | | NO: | |
| | | | | | | | | | 65) | |
| siLNA | GP6 | GPVI | In | | Anti- | Anti- | AGAAA | CACAU | AAGA | ACAUA |
| | | | vivo | | throm- | throm- | GAGGC | ACUGG | AAGA | CUGGC |
| | | | | | botic - | botic - | CAGUA | CCUCU | GGCC | CUCUU |
| | | | | | chronic | acute | UGUGG | UUCUU | AGUA | UCUUU |
| | | | | | | | U | U(SEQ | UGUG | U(SEQ |
| | | | | | | | (SEQ | ID | G | ID |
| | | | | | | | ID | NO: | (SEQ | NO: |
| | | | | | | | NO: | 34) | ID | 92) |
| | | | | | | | 2) | | NO: | |
| | | | | | | | | | 66) | |
| siLNA | FCGR2A | FC- | In | | Anti- | Immune | AAUGA | CCACA | UACA | CCAAA |
| | | gamma- | vivo | | throm- | thrombo- | GAUUC | AUGGG | UUCU | UGUCU |
| | | RIIA | | | botic - | cyto- | CCAUU | AAUCU | GAGA | CAGAA |
| | | | | | acute | penia/ | GUGGA | CAUUU | CAUU | UGUAU |
| | | | | | or | immuno- | A | U(SEQ | UGGG | U(SEQ |
| | | | | | chronic | throm- | (SEQ | ID | U | ID |
| | | | | | | bosis | ID | NO: | (SEQ | NO: |
| | | | | | | | NO: | 35) | ID | 93) |
| | | | | | | | 3) | | NO: | |
| | | | | | | | | | 67) | |
| siLNA | CLEC1B | CLEC2 | In | | Anti- | Anti- | UGAAA | GAACU | AAUA | GACAC |
| | | | vivo | | throm- | throm- | GUGCC | AAAGG | UCUC | AAACU |
| | | | | | botic - | botic - | CUUUA | GCACU | CAGU | GGAGA |
| | | | | | chronic | acute | GUUCU | UUCAU | UUGU | UAUUU |
| | | | | | | | G | U(SEQ | GUCA | U(SEQ |
| | | | | | | | (SEQ | ID | C | ID |
| | | | | | | | ID | NO: | (SEQ | NO: |
| | | | | | | | NO: | 36) | ID | 94) |
| | | | | | | | 4) | | NO: | |
| | | | | | | | | | 68) | |
| SILNA | ADAM17 | ADAM17 | Platelet | | Platelet | | UAAAG | CUACG | AAUA | CCAAA |
| | | | storage | | storage | | AGAGA | AUAUU | ACCA | AGUGU |
| | | | | | lesion | | AUAUC | CUCUC | CACA | GUGGU |
| | | | | | | | GUAGU | UUUAU | CUUU | UAUUU |
| | | | | | | | C(SEQ | U(SEQ | UGGA | U(SEQ |
| | | | | | | | ID | ID | G | ID |
| | | | | | | | NO: | NO: | (SEQ | NO: |
| | | | | | | | 5) | 37) | ID | 95) |
| | | | | | | | | | NO: | |
| | | | | | | | | | 69) | |
| siLNA | ADAM10 | ADAM10 | Platelet | | Platelet | | AAAUG | CCAUU | UCUU | CAUGU |
| | | | storage | | storage | | ACAGA | UCACU | CAUA | AUGCU |
| | | | | | lesion | | GUGAA | CUGUC | CAGC | GUAUG |
| | | | | | | | AUGGC | AUUUU | AUAC | AAGAU |
| | | | | | | | A(SEQ | U(SEQ | AUGG | U(SEQ |
| | | | | | | | ID | ID | C | ID |
| | | | | | | | NO: | NO: | (SEQ | NO: |
| | | | | | | | 6) | 38) | ID | 96) |
| | | | | | | | | | NO: | |
| | | | | | | | | | 70) | |
| siLNA | RGS10 | RGS10 | Platelet | In | Platelet | Hemo- | UAACA | GUUUA | UUCC | CCUGG |
| | | | storage | vivo | storage | stasis | AAGCA | UCAUU | AGAA | AGAAU |
| | | | | | lesion | | AUGAU | GCUUU | GAUU | CUUCU |
| | | | | | | | AAACC | GUUAU | CUCC | GGAAU |
| | | | | | | | C(SEQ | U(SEQ | AGGG | U(SEQ |
| | | | | | | | ID | ID | A | ID |
| | | | | | | | NO: | NO: | (SEQ | NO: |
| | | | | | | | 7) | 39) | ID | 97) |
| | | | | | | | | | NO: | |
| | | | | | | | | | 71) | |
| SiLNA | RGS18 | RGS18 | Platelet | In | Platelet | Hemo- | AUGAA | GCAGA | UGUA | GGAAC |
| | | | storage | vivo | storage | stasis | ACUCA | AACCU | UAAC | AAGAC |
| | | | | | lesion | | GGUUU | GAGUU | UGUC | AGUUA |
| | | | | | | | CUGCA | UCAUU | UUGU | UACAU |
| | | | | | | | C(SEQ | U(SEQ | UCCA | U(SEQ |
| | | | | | | | ID | ID | U | ID |
| | | | | | | | NO: | NO: | (SEQ | NO: |
| | | | | | | | 8) | 40) | ID | 98) |
| | | | | | | | | | NO: | |
| | | | | | | | | | 72) | |
| siLNA | GRK2 | GRK2 | Platelet | In | Platelet | Hemo- | UGUAU | GAGAU | AAAU | CCAUA |
| | | | storage | vivo | storage | stasis | GAGUC | CUUCG | CUCU | CAUCG |
| | | | | | lesion | | GAAGA | ACUCA | UCGA | AAGAG |
| | | | | | | | UCUCC | UACAU | UGUA | AUUUU |
| | | | | | | | C(SEQ | U(SEQ | UGGC | U(SEQ |
| | | | | | | | ID | ID | U | ID |
| | | | | | | | NO: | NO: | (SEQ | NO: |
| | | | | | | | 9) | 41) | ID | 99) |
| | | | | | | | | | NO: | |
| | | | | | | | | | 73) | |
| siLNA | GRK5 | GRK5 | Platelet | In | Platelet | Hemo- | UAAUG | CUGAA | UUCA | CCUUC |
| | | | storage | vivo | storage | stasis | UGAGG | GUUCC | UGUU | UUCAG |
| | | | | | lesion | | GAACU | CUCAC | CCUG | GAACA |
| | | | | | | | UCAGG | AUUAU | AAGA | UGAAU |
| | | | | | | | A(SEQ | U(SEQ | AGGG | U(SEQ |
| | | | | | | | ID | ID | G | ID |
| | | | | | | | NO: | NO: | (SEQ | NO: |
| | | | | | | | 10) | 42) | ID | 100) |
| | | | | | | | | | NO: | |
| | | | | | | | | | 74) | |
| siLNA | GRK6 | GRK6 | Platelet | In | Platelet | Hemo- | UUUUU | GCAAG | UCUU | GAGAA |
| | | | storage | vivo | storage | stasis | CUCUA | AAGCU | GAUC | AAAGC |
| | | | | | lesion | | GCUUC | AGAGA | CGCU | GGAUC |
| | | | | | | | UUGCA | AAAAU | UUUU | AAGAU |
| | | | | | | | G(SEQ | U(SEQ | CUCU | U(SEQ |
| | | | | | | | ID | ID | A | ID |
| | | | | | | | NO: | NO: | (SEQ | NO: |
| | | | | | | | 11) | 43) | ID | 101) |
| | | | | | | | | | NO: | |
| | | | | | | | | | 75) | |
| siLNA | CXCL4 | PF4 | In | | Anti- | Immune | AUCUU | CGCUG | UUUC | GCUGA |
| | | | vivo | | throm- | thrombo- | CUUCA | AAGCU | CUUC | AGAAU |
| | | | | | botic - | cyto- | GCUUC | GAAGA | CAUU | GGAAG |
| | | | | | acute | penia/ | AGCGC | AGAUU | CUUC | GAAAU |
| | | | | | or | immuno- | U(SEQ | U(SEQ | AGCG | U(SEQ |
| | | | | | chronic | throm- | ID | ID | U | ID |
| | | | | | | bosis | NO: | NO: | (SEQ | NO: |
| | | | | | | | 12) | 44) | ID | 102) |
| | | | | | | | | | NO: | |
| | | | | | | | | | 76) | |
| siLNA | ESR2 | ERbeta | In | In | Anti- | Anti- | AUACA | CCAGU | AAUU | GAAAU |
| | | | vivo | vivo | throm- | throm- | GAUGU | UAUCA | CCUU | GCGUA |
| | | | | | botic - | botic - | GAUAA | CAUCU | CUAC | GAAGG |
| | | | | | chronic | acute | CUGGC | GUAUU | GCAU | AAUUU |
| | | | | | | | G(SEQ | U(SEQ | UUCC | U(SEQ |
| | | | | | | | ID | ID | C | ID |
| | | | | | | | NO: | NO: | (SEQ | NO: |
| | | | | | | | 13) | 45) | ID | 103) |
| | | | | | | | | | NO: | |
| | | | | | | | | | 77) | |
| siLNA | ANO6 | TMEM16F | In | | Anti- | Anti- | AACAA | GCGAA | UCUU | GCAGU |
| | | | vivo | | throm- | throm- | AGUCA | GAAUU | GUUG | UAGAA |
| | | | | | botic - | botic - | AUUCU | GACUU | CUUC | GCAAC |
| | | | | | chronic | acute | UCGCU | UGUUU | UAAC | AAGAU |
| | | | | | | | G(SEQ | U(SEQ | UGCA | U(SEQ |
| | | | | | | | ID | ID | G | ID |
| | | | | | | | NO: | NO: | (SEQ | NO: |
| | | | | | | | 14) | 46) | ID | 104) |
| | | | | | | | | | NO: | |
| | | | | | | | | | 78) | |
| siLNA | F2R | PARI | In | | Anti- | Anti- | AAGCA | GCAUC | UUUC | CAUGA |
| | | | vivo | | throm- | throm- | AAUGA | UUCAU | AUUG | UGUGC |
| | | | | | botic - | botic - | UGAAG | CAUUU | AGCA | UCAAU |
| | | | | | chronic | acute | AUGCA | GCUUU | CAUC | GAAAU |
| | | | | | | | G(SEQ | U(SEQ | AUGA | U(SEQ |
| | | | | | | | ID | ID | C | ID |
| | | | | | | | NO: | NO: | (SEQ | NO: |
| | | | | | | | 15) | 47) | ID | 105) |
| | | | | | | | | | NO: | |
| | | | | | | | | | 79) | |
| siLNA | F2RL3 | PAR4 | In | | Anti- | Anti- | UUCAU | CCACC | AUAC | ACUCU |
| | | | vivo | | throm- | throm- | CAGCA | AUGCU | AUGU | AUGGU |
| | | | | | botic - | botic - | GCAUG | GCUGA | GACC | CACAU |
| | | | | | chronic | acute | GUGGA | UGAAU | AUAG | GUAUU |
| | | | | | | | G(SEQ | U(SEQ | AGUG | U(SEQ |
| | | | | | | | ID | ID | C | ID |
| | | | | | | | NO: | NO: | (SEQ | NO: |
| | | | | | | | 16) | 48) | ID | 106) |
| | | | | | | | | | NO: | |
| | | | | | | | | | 80) | |
| sILNA | ITGA2B | GPIIB | In | | Anti- | Anti- | UCAUU | GCAUU | ACUG | CGUAU |
| | | | vivo | | throm- | throm- | UUCCA | UACGU | AAUG | UUUGG |
| | | | | | botic - | botic - | CGUAA | GGAAA | CCCA | GCAUU |
| | | | | | chronic | acute | AUGCG | AUGAU | AAAU | CAGUU |
| | | | | | | | G(SEQ | U(SEQ | ACGA | U(SEQ |
| | | | | | | | ID | ID | C | ID |
| | | | | | | | NO: | NO: | (SEQ | NO: |
| | | | | | | | 17) | 49) | ID | 107) |
| | | | | | | | | | NO: | |
| | | | | | | | | | 81) | |
| siLNA | ITGB3 | GPIIIA | In | | Anti- | Anti- | AUAAG | GCUCA | UGUU | CCAUG |
| | | | vivo | | throm- | throm- | CAUCA | UUGUU | UGUA | UUUGG |
| | | | | | botic - | botic - | ACAAU | GAUGC | GCCA | CUACA |
| | | | | | chronic | acute | GAGCU | UUAUU | AACA | AACAU |
| | | | | | | | G(SEQ | U(SEQ | UGGG | U(SEQ |
| | | | | | | | ID | ID | C | ID |
| | | | | | | | NO: | NO: | (SEQ | NO: |
| | | | | | | | 18) | 50) | ID | 108) |
| | | | | | | | | | NO: | |
| | | | | | | | | | 82) | |
| siLNA | RHOB | RHOB | Platelet | | Platelet | Anti- | UCAAU | ACUAU | UAGU | CCACC |
| | | | storage | | storage | inflam- | GUCGG | GUGGC | UCUC | GUCUU |
| | | | | | lesion | matory- | CCACA | CGACA | GAAG | CGAGA |
| | | | | | | | UAGUU | UUGAU | ACGG | ACUAU |
| | | | | | | | C(SEQ | U(SEQ | UGGG | U(SEQ |
| | | | | | | | ID | ID | C | ID |
| | | | | | | | NO: | NO: | (SEQ | NO: |
| | | | | | | | 19) | 51) | ID | 109) |
| | | | | | | | | | NO: | |
| | | | | | | | | | 83) | |
| siLNA | IL1B | IL1B | In | Platelet | Anti- | | UCUUC | CCUUC | UCUU | CGAUU |
| | | | vivo | storage | inflam- | | UUCAA | AUCUU | GUUG | UGUCU |
| | | | | | matory- | | AGAUG | UGAAG | AAGA | UCAAC |
| | | | | | | | AAGGG | AAGAU | CAAA | AAGAU |
| | | | | | | | A(SEQ | U(SEQ | UCGC | U(SEQ |
| | | | | | | | ID | ID | U | ID |
| | | | | | | | NO: | NO: | (SEQ | NO: |
| | | | | | | | 20) | 52) | ID | 110) |
| | | | | | | | | | NO: | |
| | | | | | | | | | 84) | |
| siLNA | NLRP3 | NLRP3 | In | Platelet | Anti- | | ACUUU | CGUAA | AGAU | CCAAG |
| | | | vivo | storage | inflam- | | CUGUA | GAAGU | UGAU | AUUGA |
| | | | | | matory- | | CUUCU | ACAGA | CUCA | GAUCA |
| | | | | | | | UACGG | AAGUU | AUCU | AUCUU |
| | | | | | | | U(SEQ | U(SEQ | UGGG | U(SEQ |
| | | | | | | | ID | ID | G | ID |
| | | | | | | | NO: | NO: | (SEQ | NO: |
| | | | | | | | 21) | 53) | ID | 111) |
| | | | | | | | | | NO: | |
| | | | | | | | | | 85) | |
| siLNA | RHOA | RHOA | Platelet | In | Platelet | Throm- | ACAAU | GGAAG | AACA | GAAGA |
| | | | storage | vivo | storage | bocyto- | CACCA | AAACU | AUCA | AACUG |
| | | | | | lesion | penia | GUUUC | GGUGA | CCAG | GUGAU |
| | | | | | | | UUCCG | UUGUU | UUUC | UGUUU |
| | | | | | | | G(SEQ | U(SEQ | UUCC | U(SEQ |
| | | | | | | | ID | ID | G | ID |
| | | | | | | | NO: | NO: | (SEQ | NO: |
| | | | | | | | 22) | 54) | ID | 112) |
| | | | | | | | | | NO: | |
| | | | | | | | | | 86) | |
| siLNA | NEU1 | NEU1 | Platelet | In | Platelet | Throm- | AAUAU | GAAUC | CAUA | GCCAA |
| | | | storage | vivo | storage | bocyto- | CCAGG | UCUCC | GACA | AAUCA |
| | | | | | lesion | penia | GAGAG | CUGGA | CUGA | GUGUC |
| | | | | | | | AUUCC | UAUUU | UUUU | UAUGU |
| | | | | | | | G(SEQ | U(SEQ | GGCC | U(SEQ |
| | | | | | | | ID | ID | A | ID |
| | | | | | | | NO: | NO: | (SEQ | NO: |
| | | | | | | | 23) | 55) | ID | 113) |
| | | | | | | | | | NO: | |
| | | | | | | | | | 87) | |
| siLNA | RASA3 | RASA3 | Platelet | In | Platelet | Hemo- | UCAAA | GCAAG | UCAU | CUUAA |
| | | | storage | vivo | storage | stasis | GUGGG | AAGUC | GAAC | AGAAG |
| | | | | | lesion | | ACUUC | CCACU | CCUU | GGUUC |
| | | | | | | | UUGCU | UUGAU | CUUU | AUGAU |
| | | | | | | | G(SEQ | U(SEQ | AAGC | U(SEQ |
| | | | | | | | ID | ID | A | ID |
| | | | | | | | NO: | NO: | (SEQ | NO: |
| | | | | | | | 24) | 56) | ID | 114) |
| | | | | | | | | | NO: | |
| | | | | | | | | | 88) | |
| siLNA | GNAS | Gαs | In | Platelet | Anti- | Hemo- | AAAGU | GAAGA | ACUC | GAAGC |
| | | | vivo | storage | bleed- | stasis | AGUCC | UUGAG | AAAU | UAUGC |
| | | | | | ing/ | | UCAAU | GACUA | GGCA | CAUUU |
| | | | | | pro- | | CUUCG | CUUUU | UAGC | GAGUU |
| | | | | | hemo- | | A(SEQ | U(SEQ | UUCT | U(SEQ |
| | | | | | static | | ID | ID | T | ID |
| | | | | | | | NO: | NO: | (SEQ | NO: |
| | | | | | | | 25) | 57) | ID | 115) |
| | | | | | | | | | NO: | |
| | | | | | | | | | 89) | |
| siLNA | PTGIR | IP | In | Platelet | Anti- | Hemo- | UGAGC | GGCCU | AAGA | CGUCC |
| | | | vivo | storage | bleed- | stasis | AUGGA | GGCGU | GGAU | AUGCU |
| | | | | | ing/ | | CGCCA | CCAUG | GAGC | CAUCC |
| | | | | | pro- | | GGCCG | CUCAU | AUGG | UCUUU |
| | | | | | hemo- | | A(SEQ | U(SEQ | ACGC | U(SEQ |
| | | | | | static | | ID | ID | C | ID |
| | | | | | | | NO: | NO: | (SEQ | NO: |
| | | | | | | | 26) | 58) | ID | 116) |
| | | | | | | | | | NO: | |
| | | | | | | | | | 90) | |
| SILNA | GSK3B | GSK3β | In | Platelet | Anti- | Hemo- | AAAUG | GAUUG | | |
| | | | vivo | storage | bleed- | stasis | AUCCA | GAAAU | | |
| | | | | | ing/ | | UUUCC | GGAUC | | |
| | | | | | pro- | | AAUCA | AUUUU | | |
| | | | | | hemo- | | C(SEQ | U(SEQ | | |
| | | | | | static | | ID | ID | | |
| | | | | | | | NO: | NO: | | |
| | | | | | | | 27) | 59) | | |
| SILNA | PPIA | CYPA | In | | Anti- | Anti- | AGACG | GAGCC | | |
| | | | vivo | | throm- | throm- | CGGCC | CUUGG | | |
| | | | | | botic - | botic - | CAAGG | GCCGC | | |
| | | | | | chronic | acute | GCUCG | GUCUU | | |
| | | | | | | | T(SEQ | U(SEQ | | |
| | | | | | | | ID | ID | | |
| | | | | | | | NO: | NO: | | |
| | | | | | | | 28) | 60) | | |
| miR- | Multi- | Multi- | In | Platelet | Hemo- | Anti- | UGUCA | GGGUA | | |
| 223- | ple | ple | vivo | storage | stasis | inflam- | GUUUG | UUUGA | | |
| 3p | | | | | | matory, | UCAAA | CAAAC | | |
| | | | | | | hemo- | UACCC | UGACA | | |
| | | | | | | stasis | CA | UU | | |
| | | | | | | | (SEQ | (SEQ | | |
| | | | | | | | ID | ID | | |
| | | | | | | | NO: | NO: | | |
| | | | | | | | 29) | 61) | | |
| miR- | Multi- | Multi- | Platelet | In | Platelet | Anti- | GUCCA | GGAUU | | |
| 145- | ple | ple | storage | vivo | storage | throm- | GUUUU | CCUGG | | |
| 5p | | | | | lesion | botic - | CCCAG | GAAAA | | |
| | | | | | | acute | GAAUC | CUGGU | | |
| | | | | | | or | CCU | U(SEQ | | |
| | | | | | | chronic | (SEQ | ID | | |
| | | | | | | | ID | NO: | | |
| | | | | | | | NO: | 62) | | |
| | | | | | | | 30) | | | |
| miR- | Multi- | Multi- | Mega- | | Platelet | | UCUCC | CUGGU | | |
| 150 | ple | ple | kary- | | produc- | | CAACC | ACAAG | | |
| | Multi- | Multi- | ocyte | | tion | | CUUGU | GGUUG | | |
| | | | culture | | | | ACCAG | GGAGA | | |
| | | | | | | | UG | UU | | |
| | | | | | | | (SEQ | (SEQ | | |
| | | | | | | | ID | ID | | |
| | | | | | | | NO: | NO: | | |
| | | | | | | | 31) | 63) | | |
| miR- | Multi- | Multi- | Mega- | | Platelet | | UCCCU | ACAAG | | |
| 125b- | ple | ple | kary- | | produc- | | GAGAC | UUAGG | | |
| 2 | | | ocyte | | tion | | CCUAA | GUCUC | | |
| | | | culture | | | | CUUGU | AGGGA | | |
| | | | | | | | GA | UU | | |
| | | | | | | | (SEQ | (SEQ | | |
| | | | | | | | ID | ID | | |
| | | | | | | | NO: | NO: | | |
| | | | | | | | 32) | 64) | | |

Sequence key:
Bolded italic = locked nucleic acid (LNA) modification
Bolded = 2′-OMe nucleotide or 2′-F modification.
All sequences are 5′-->3′

For Assessing Therapeutic Window for P2ry12 siLNA and Ticagrelor in Mice

Therapeutic window (TW) was assessed in age-matched wild type C57B16/J mice as the separation between antithrombotic and bleeding effects, across a 3-log dose range of either ticagrelor or P2ry12 siLNA (gene name, P2ry12; protein name, P2y12) following intravenous administration. Scrambled siLNA, with no predicted human mRNA targets, was used as control. Thrombosis was tested using FeCl₃ intimal injury to the carotid artery and Doppler monitoring of blood flow downstream from the injury site, and bleeding was tested by tail clip amputation, blood collection, and weight measurement to determine blood loss. Tests were performed in anesthetized mice at 5 min after ticagrelor administration, and 24 hrs after siLNA administration.

Thrombosis: The left carotid artery was exposed, cleaned of excess tissue, and placed on a Doppler probe linked to a blood flow monitor. The artery was raised up (temporarily cutting blood flow), and a 2×2 mm-square piece of filter paper soaked with 4 μL of 7.5% FeCl₃ was placed on the exposed carotid artery for 90 sec to induce injury. Blood flow was monitored for 20 min after the filter paper was removed. In a typical experimental setting, with no antithrombotic treatment an occlusive clot will form (thrombosis) within ˜9-11 minutes after the vascular injury, causing blockade of blood flow downstream. In some cases, treatments that reduce clot growth to a moderate degree will be observed as an incomplete restoration of blood flow through the narrowed vessel. Thrombosis is shown in squares as percent blockade of blood flow at the 20 min post-injury endpoint compared to starting flow rate, calculated from mean flow rates for the starting and final 100 sec.

Bleeding: A fresh razor blade was used to sever 1-mm diameter tip of the mouse tail, as measured by calipers, and the tail was immersed in warm saline for 20 min. The increase in weight of the saline tube after the assay provided the weight of blood loss during the assay time frame. Inhibition of hemostasis is shown in circles, calculated as blood weight loss/initial mouse weight (wt, g/g), expressed as observed fold change over the mean blood weight loss/mouse weight from vehicle controls (n=18).

Therapeutic window (TW) was calculated as the ratio of dose resulting in a 3-fold increase in blood loss compared to controls (Inhibition of hemostasis, blue dotted line) to the dose resulting in 50% restored blood flow (Thrombosis, red dashed line). Results are shown ±s.e.m. n=3-8 for each. n.s., no significant difference from controls.

### Example 1: Naked Double-Stranded miRNA Mimics Selectively Transfect Bone Marrow Megakaryocytes In Vitro

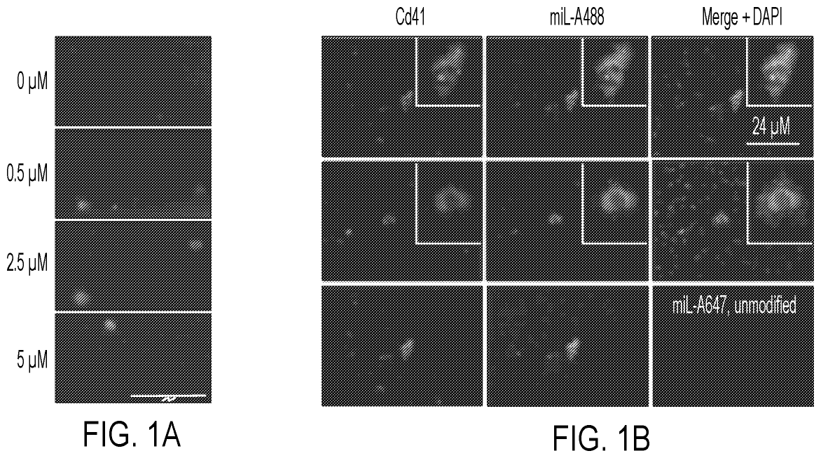

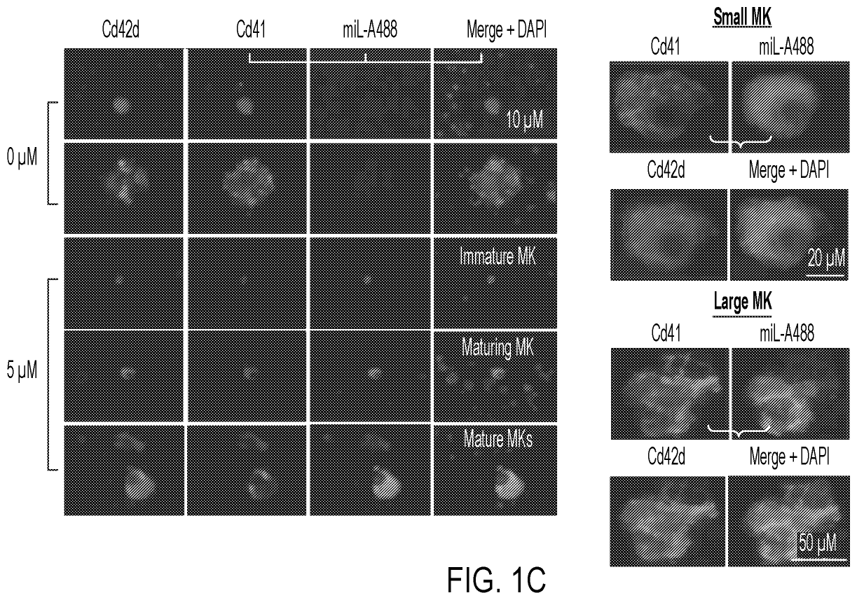

Murine bone marrow cell suspensions freshly extracted from femurs were incubated without washing (e.g., with existing plasma and ribonucleases, modeling the in vivo state) with AF488 (green)-conjugated miL-223-3p ds mimic at the indicated concentrations for 1 hour, then either imaged live or fixed and processed. Whole unfixed bone marrow cell suspensions captured on poly-L-lysine (PLL)-coated slides were imaged live for AF488 (FIG. 1A). Only megakaryocytes (MK) show green fluorescence, indicating MK specific uptake. Fixed bone marrow cells were counterstained for MK/platelet marker Cd41) and DAPI for nuclei (FIG. 1B). Only MKs, but not other bone marrow cells, show miLNA uptake. In this experiment, bone marrow cells were co-incubated with both the miLNA-AF488, and an unmodified version of miR-223-3p conjugated to AlexaFluor647. Lower panels show no fluorescence indicating degradation of the unmodified miRNA mimic. Cells as in (FIG. 1B) counterstained for two MK/platelet markers Cd42d and Cd41, showing uptake by MKs at different stages including immature growth stages (small MK) (FIG. 1C).

### Example 2: Infused Naked Double-Stranded miRNA Mimics Selectively Transfect Bone Marrow Megakaryocytes and Platelets In Vivo

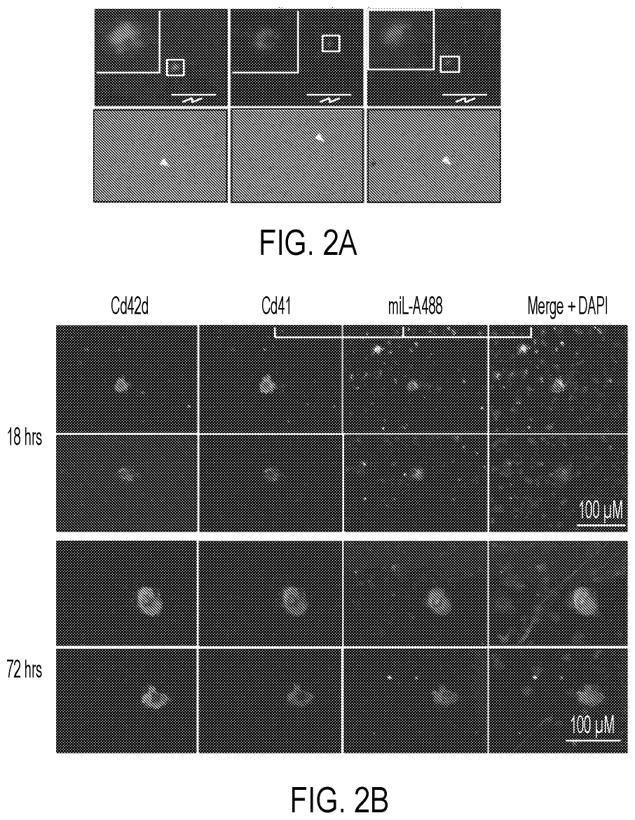

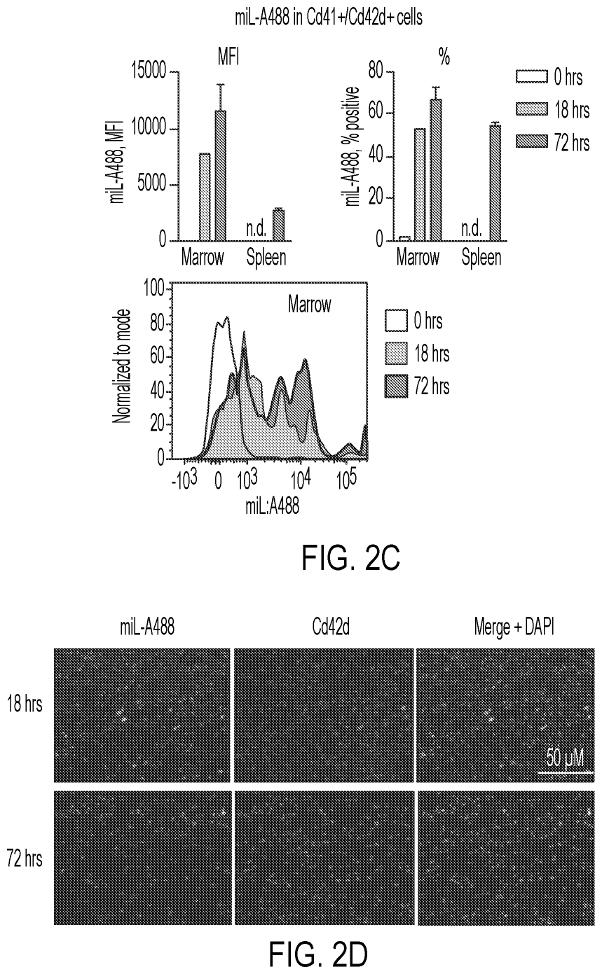

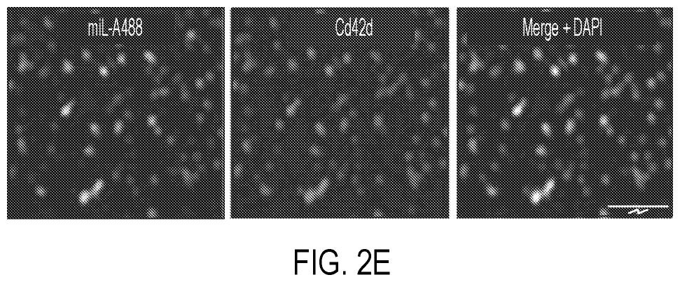

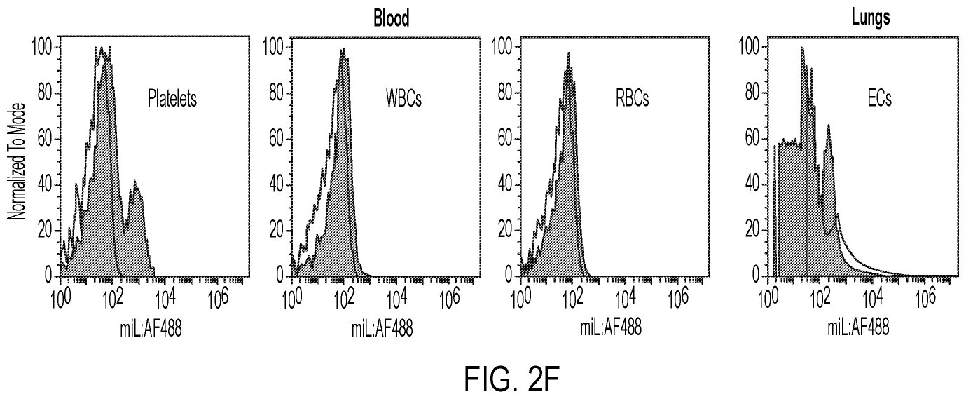

miL-223-3p conjugated to AlexaFluor488, as in (FIG. 1A), was infused into the tail vein of WT mice. Bone marrow cell suspensions were extracted and processed as in (FIGS. 1A-1C). Whole bone marrow cell suspensions extracted at 2 hrs after infusion, captured on poly-L-lysine (PLL)-coated slides and imaged live for AF488(FIG. 2A). Only megakaryocytes (MK) show green fluorescence, indicating MK-specific in vivo uptake. Fixed bone marrow cells were counterstained for MK/platelet markers Cd42d and Cd41, and DAPI for nuclei (FIG. 2B). Only MKs and platelets, but not other bone marrow cells, show miLNA uptake, at 18 hours and 72 hours post-infusion. Cells as in (FIG. 2B) analyzed by flow cytometry for miL uptake (by fluorescence), using Cd41/Cd42d and size (forward scatter FSC) to identify MKs (FIG. 2C). In this experiment spleens were also extracted and splenocyte suspensions were analyzed at 72 hours, together showing robust MK uptake and retention of the miLNA in bone marrow and splenic MKs. Peripheral blood cells from the same animals as above were fixed and captured on coverslips, and platelets were identified with Cd42d antibodies. Platelet miLNA in vivo uptake efficiency approached 100%, as had previously been observed with washed in vitro platelets (FIG. 2D).

### Example 3: Infused Naked RhoA siLNAs Suppress RhoA Protein Expression Selectively in Bone Marrow Megakaryocytes, and Modulate Megakaryocyte Development and Platelet Production In Vivo

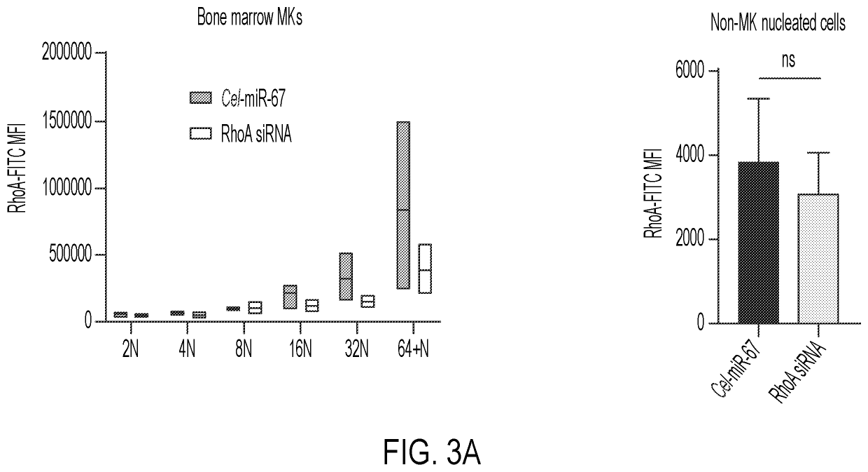

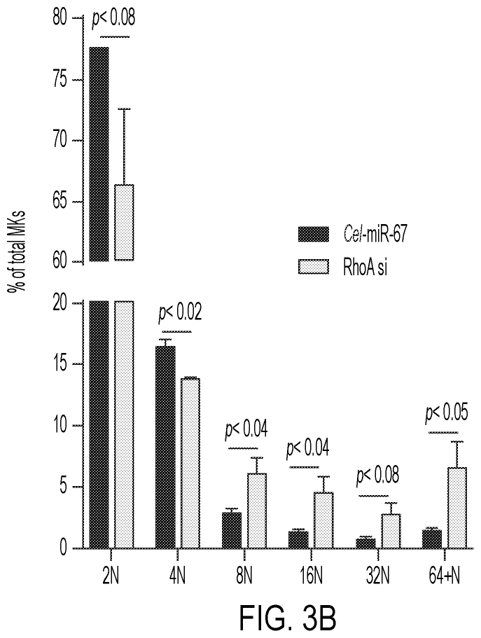

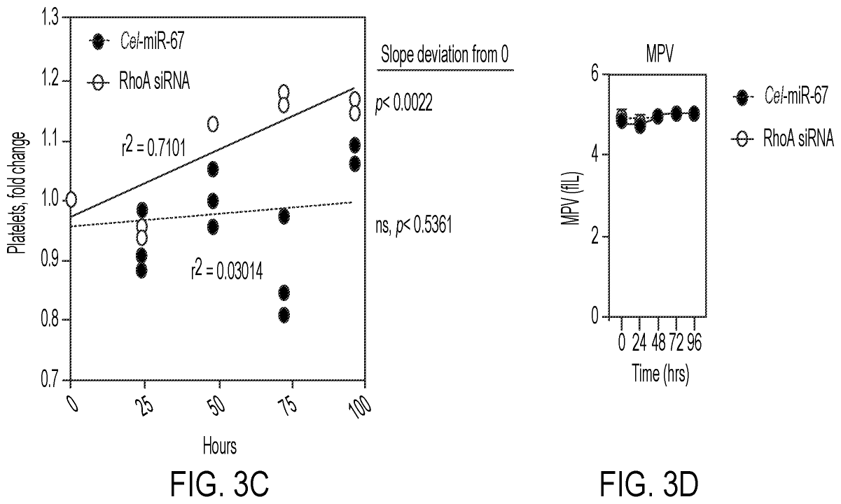

These experiments demonstrate the effects of infused naked siLNAs on megakaryocyte (MK) target gene expression and cellular function. RhoA or Cel-miL-67 siLNAs were infused into WT mice as previously detailed. Peripheral blood was sampled daily to evaluate blood cells, and bone marrow was extracted at 96 hours after start of the experiments. Cel-miL-67 was used as a negative control. RhoA siLNAs suppress RhoA protein expression selectively in bone marrow MKs after infusion (FIG. 3A). RhoA levels were measured by flow cytometry in permeablized whole bone marrow cell suspensions using RhoA antibodies, coupled with Cd42d antibodies to label MKs, and propidium iodide (PI) to label nuclear DNA. (Left panel) RhoA expression levels in MKs as a function of ploidy class determined by PI staining. RhoA expression increased in larger, more mature MKs as expected due to expanded cytoplasm, but this increase was suppressed by RhoA siLNAs. (Right panel) nucleated (PI+) non-MK bone marrow cells showed no change in RhoA protein levels. RhoA siLNAs modulate RhoA-dependent MK function (FIG. 3B). RhoA knockout mice and RhoA-inhibitor-treated mice demonstrated that acute RhoA suppression leads to increased MK ploidy. RhoA siLNAs caused increased MK ploidy compared to control siLNA, demonstrating specific functional effects of RhoA suppression in MKs in vivo. FIG. 3C shows increased peripheral blood platelet count with RhoA siLNA, corresponding to increased MK ploidy (FIG. 3B). No change to mean platelet volumes with RhoA siLNA, confirming that increased MK ploidy drives higher platelet count but not generation of larger platelets (FIG. 3D).

### Example 4: Infused Naked P2y12 (P2ry12) siLNA Reduces Platelet ADP Reactivity and Prevents Injury Induced Arterial Thrombosis in WT Mice, Phenocopying Current Frontline Antiplatelet Drugs Targeting P2Y12 (e.g., Plavix, Cangrelor, Ticagrelor)

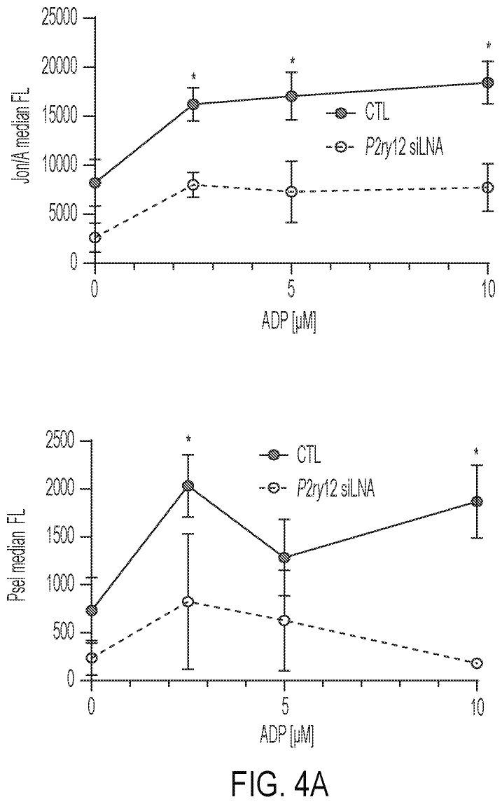

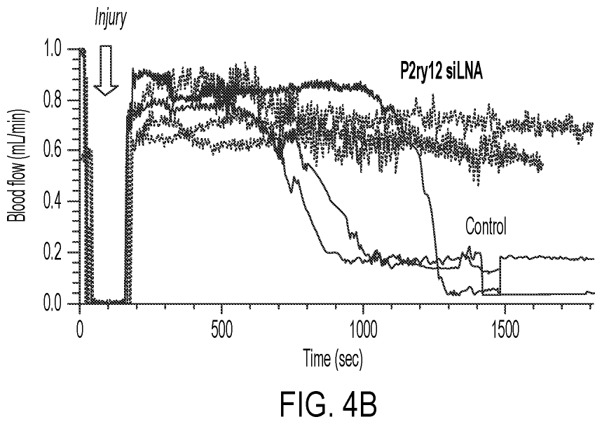

FIGS. 4A-4B show that infused naked P2y12 (P2ry12) siLNA reduces platelet ADP activation response and prevents injury induced arterial thrombosis in WT mice, phenocopying current frontline antiplatelet drugs targeting P2Y12. (FIG. 4A) P2ry12 siLNA infusion reprograms platelets with reduced ADP reactivity. Platelet ADP response was tested by flow cytometry (FACS) in whole blood cell suspensions 24 hours after P2ry12 or control scrambled (CTL) siLNA infusion, for ADP-induced GpIIb/IIIa integrin activation with JonA antibodies (upper panel) and alpha-granule secretion with P-selectin antibodies (lower panel). p<0.05. n=3. (FIG. 4B) WT mice were infused by tail vein injection with either saline (control) or naked P2ry12 siLNA at 0.2 mg/kg, daily for 4 days. At 96 hours (day 5), anesthetized mice were subjected to arterial injury to the carotid artery by application of filter paper soaked in 4 μL of a solution of 7.5% FeCl₃ for 30 seconds (“Injury”), followed by removal of the paper and restoration of blood flow. Blood flow was monitored with a Doppler probe downstream of the injury site for up to 30 minutes after injury.

As shown in FIG. 4B, arteries occluded, blocking blood flow, in control mice between 5-12 minutes after injury, as expected. In contrast, arteries in mice that had received naked P2ry12 siLNA (targeting the P2y12 ADP receptor, the target of current frontline antiplatelet antithrombotic drugs) remained patent and no occlusion was observed over the experimental time frame. n=3 each.

### Example 5: Single Injection of Naked miL-223-3p miRNA Mimic Transiently and Potently Increases Platelet ADP Reactivity

These experiments demonstrate the effects of infused naked miLNAs on circulating platelets. miR-223-3p (miL-223-3p) was used as it was found that this miRNA targets key negative regulators of platelet response to ADP agonist; importantly, these targets have rapid turnover in platelets and must be constitutively translated. It was hypothesized that miR-223-3p overexpression with miL-223-3p would lead to direct platelet effects—independent of MK effects—by suppression of its target negative regulator proteins in blood platelets.

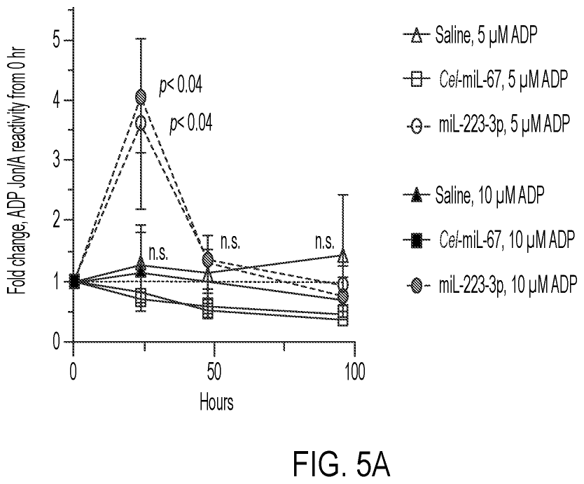

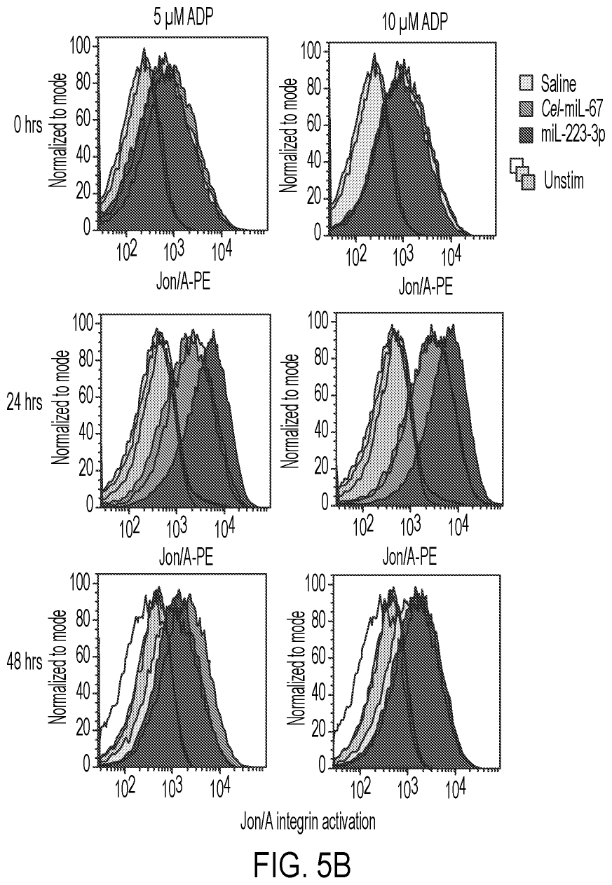

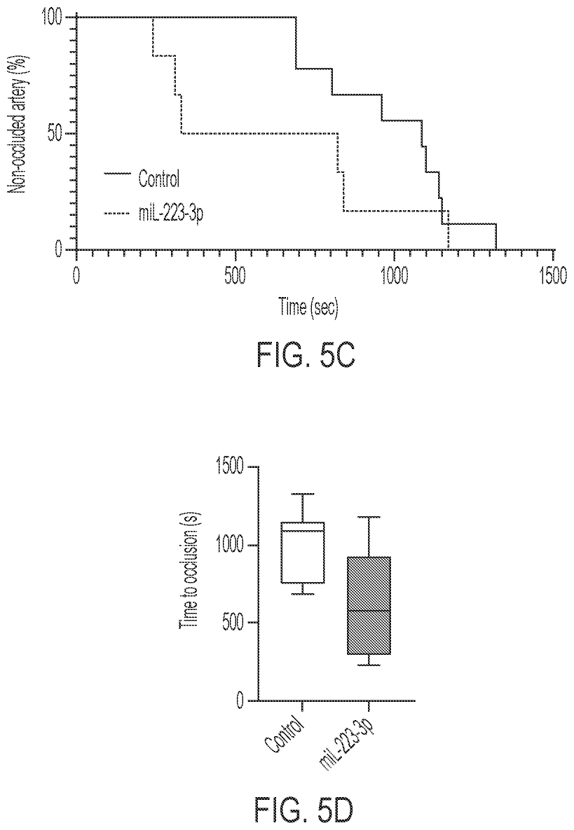

As shown in FIG. 5A, miL-223-3p injection led to a 4-fold mean increase in platelet ADP reactivity assessed 24 hours post-injection, as measured by flow cytometry for Jon/A antibodies recognizing the activated form of integrin GpIIb/IIIa, whereas reactivity was unaffected by control Cel-miL-67 or saline infusion. The increased reactivity was transient, indicating consumption of the targeting miLNA and reconstitution of the targeted proteins at later times. As shown in FIG. 5B, the entire population of circulating platelets showed a rightward shift in ADP reactivity. This result suggests potent direct effects on circulating platelets, but effects on MKs cannot be ruled out.

### Example 6: Single or Repeat Addition of Naked ADAM17 siRNA to Platelets in Storage Concentrates Protects Against Loss of GP1bα

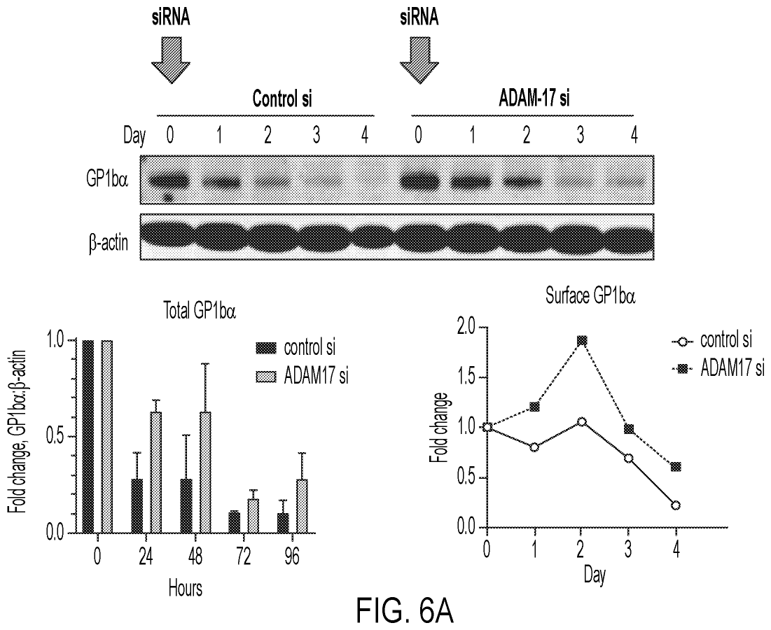

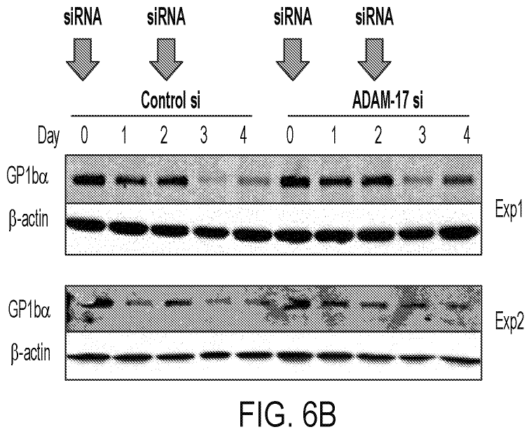

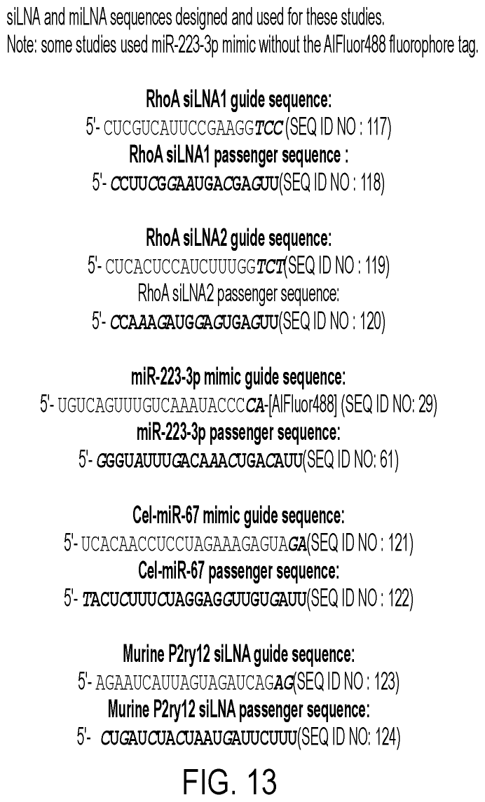

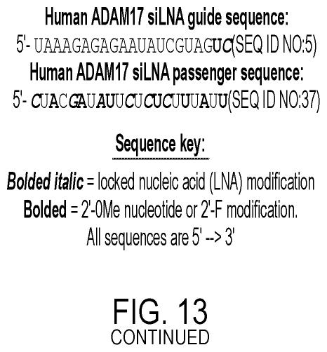

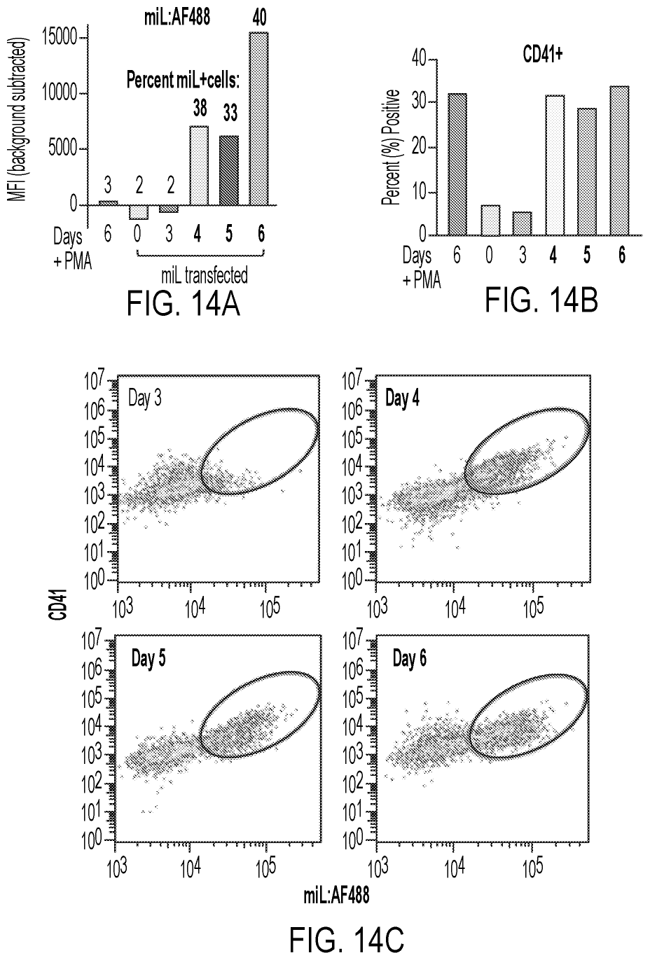

For initial testing, ADAM17 or control (Cel-miR-67) siRNAs (unmodified; Cel-miR-67 sequence is identical to the Cel-miL-67 sequence used elsewhere) were obtained from Dharmacon and added to freshly isolated, washed human platelets for 1 hr (as previously reported), followed by reconstitution with reserved autologous plasma to generate platelet-rich plasma, which was then maintained for up to 96 hours at room temperature with gentle agitation reflecting common storage conditions for platelet concentrates for transfusion recipients. ADAM17 metalloproteinase is known to cause progressive proteolytic cleavage of the GP1bα vWF receptor on platelets, leading to reduced hemostatic function in stored platelets—this is a key component of the platelet storage lesion. It was hypothesized that ADAM17 siRNA would protect against this degradation. Note: platelet counts remained equivalent over the experimental time frame (results not shown). FIG. 6A shows that a single addition of naked ADAM17 siRNA directly to the platelet storage concentrates consistently maintained GP1bα total and surface expression in stored platelets for up to 48 hours. GP1bα levels after 48 hours dropped to similar low levels as with control siRNA, indicating turnover of ADAM17 and GP1bα. FIG. 6B shows that multiple additions of ADAM17 siRNA to the stored platelet concentrates (by precipitating and rinsing the platelets, repeating transfection, and then reconstituting with reserved plasma), given at 0 and again at 48 hours, provided prolonged maintenance of GP1bα in the stored platelets. Based on these data, an ADAM17 siLNA (FIG. 13) was designed and generated.

### Example 7: Single Addition of Naked ADAM17 siLNA to Platelet Storage Concentrates Protects Against Loss of GP1bα Function

ADAM17 or control (Cel-miL-67) siLNA (modified as shown in FIG. 13) was added directly to freshly isolated human platelet-rich plasma, which was then maintained for up to 96 hours at room temperature with gentle agitation reflecting common storage conditions for platelet concentrates for transfusion recipients.

At the indicated times, a fraction of the platelet-rich plasma was moved to an aggregometer at 37° C. and given the GP1bα cofactor ristocetin (Rs) at 0.5, 1.0 or 1.5 mg/mL to initiate GP1b-dependent platelet agglutination by crosslinking plasma vWF, which was monitored over time by light transmission. Another fraction was separated for Western blots to evaluate GP1bα expression levels in platelets.

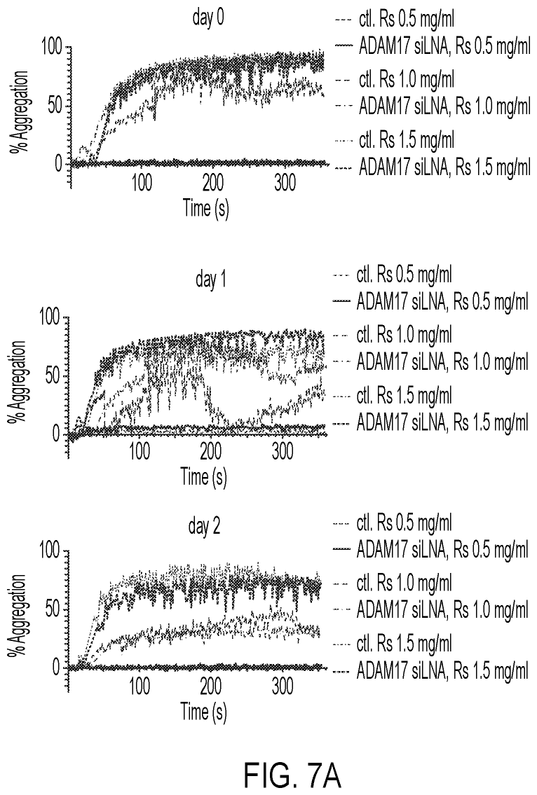

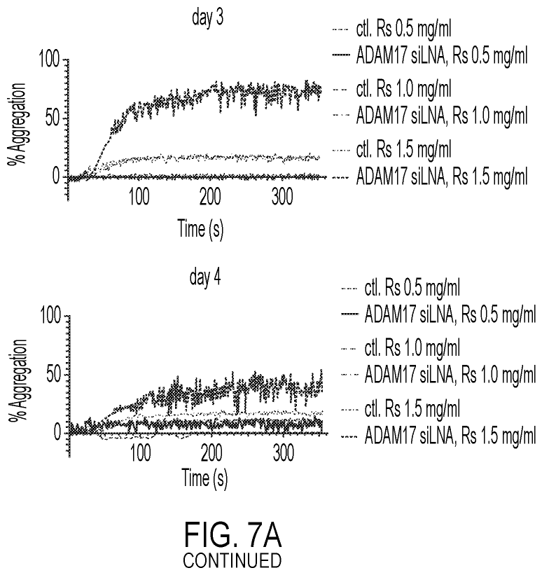

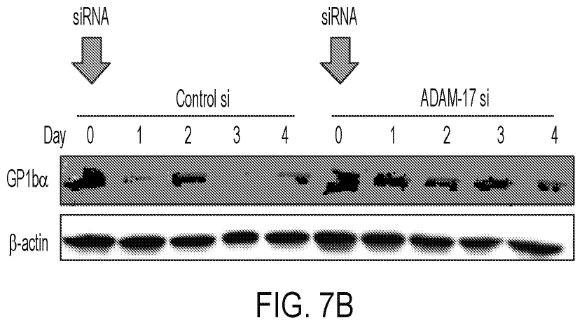

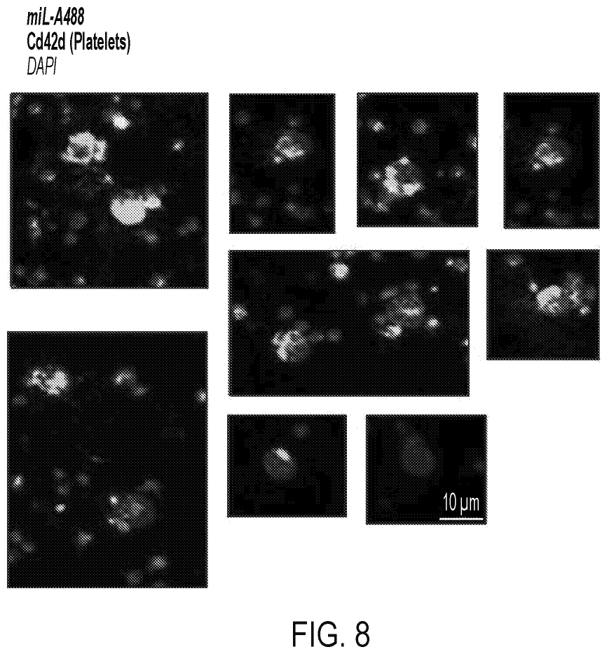

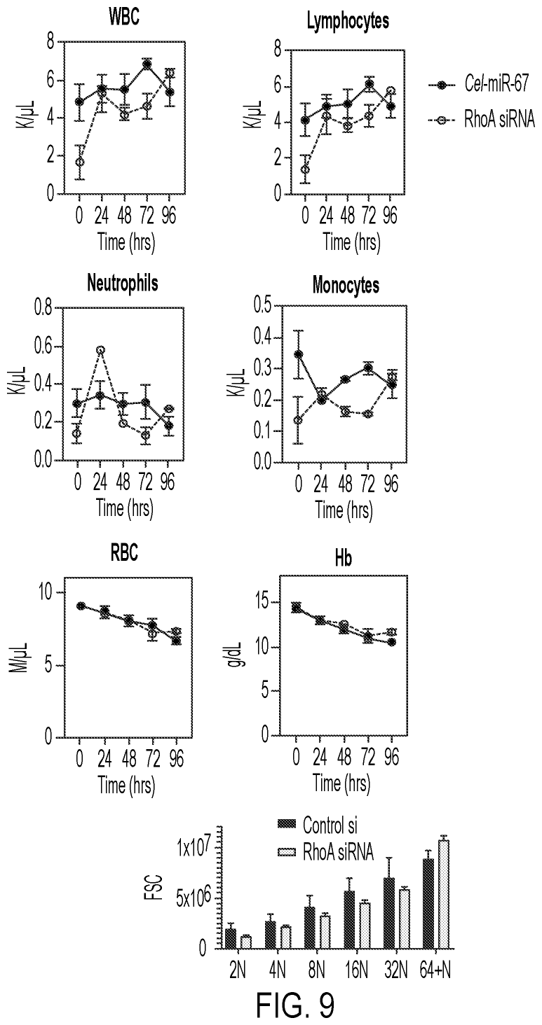

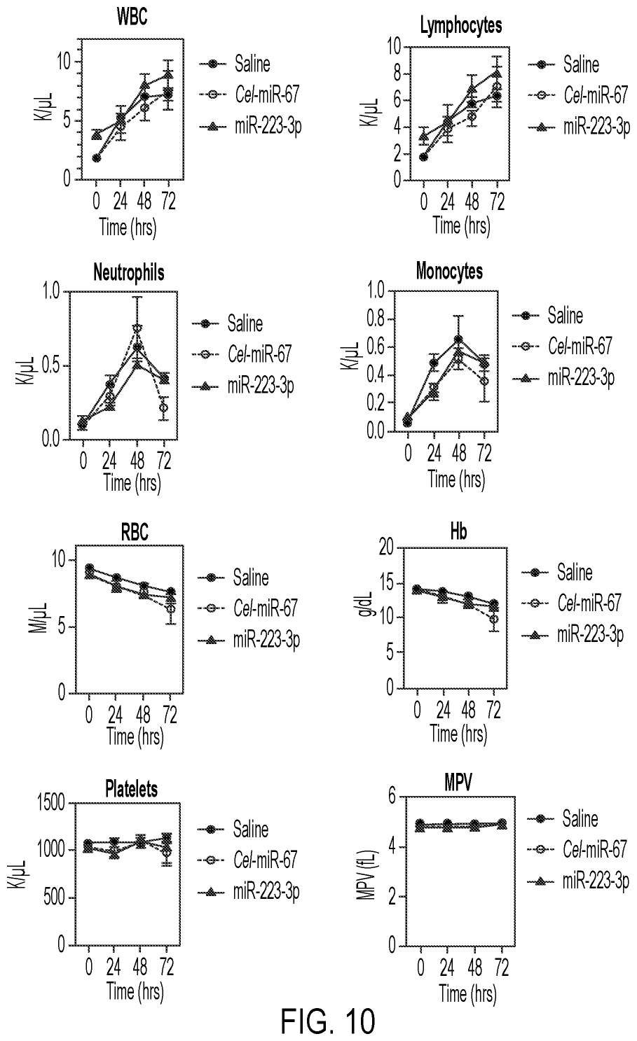

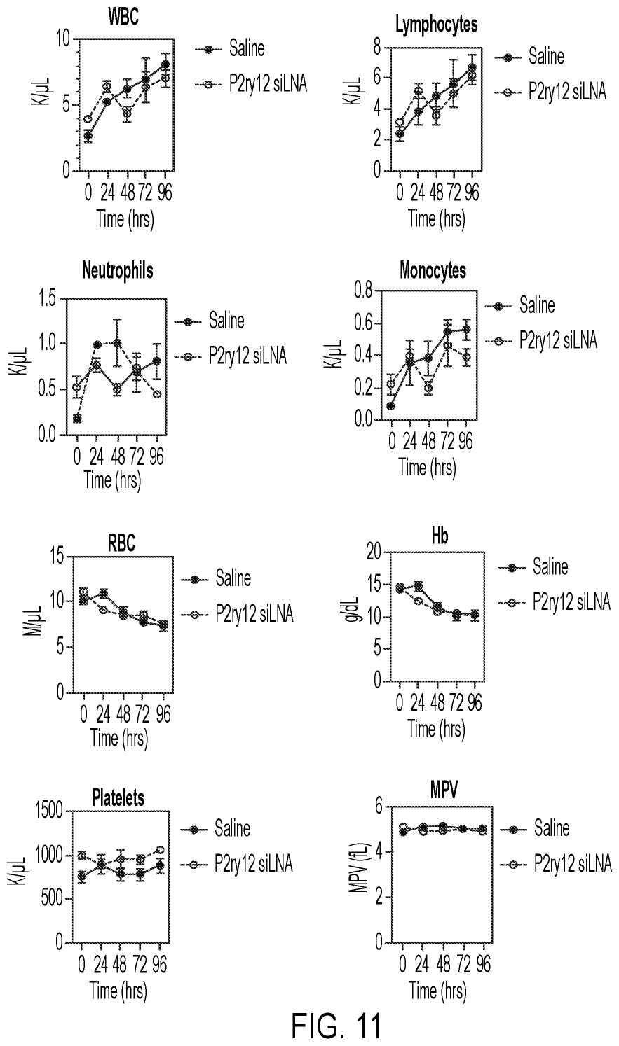

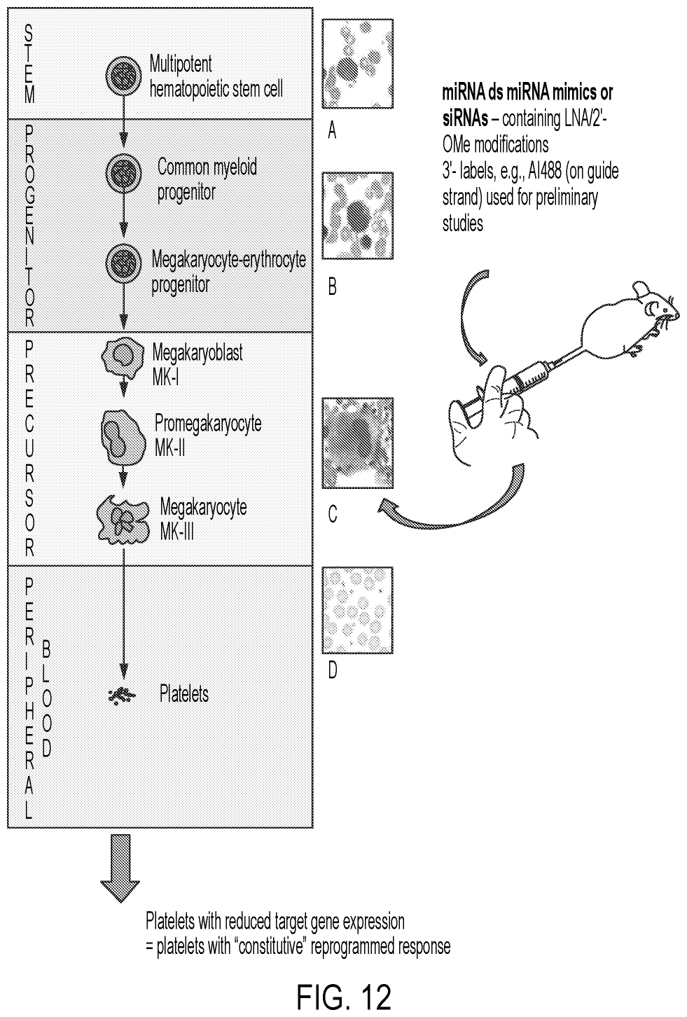

FIG. 7A shows percent platelet agglutination (aggregation) over time following addition of ristocetein (Rs) at the indicated concentrations. A single addition of naked ADAM17 siLNA directly to the platelet storage concentrates on day 0, maintained GP1bα functional reactivity over the 5-day experimental time frame. These data provide the first demonstration of improvement of cellular physiological function in stored platelets by this approach. FIG. 7B GP1bα expression in the stored platelets. These results support the use of naked siLNAs to modulate against platelet storage lesion. It is anticipated that precisely timed addition of key targeting siLNAs will yield platelets with reactivity profiles similarly to freshly isolated platelets, timed for maximum hemostatic efficacy in the transfusion recipients.

### Example 8: Therapeutic Window (TW) for P2ry12 siLNA and Ticagrelor in Mice

In the first series of experiments, an siLNA was tested against murine P2y12 (mRNA, P2ry12; P2Y12 in humans), the primary ADP receptor on platelets (also expressed in some other cells) that is responsible for so-called “secondary” feedback stimulation through autocrine and paracrine signaling after partially-stimulated platelets, responding to other stimuli, secrete ADP which then stimulates the secondary response through P2Y12. The ADP-driven P2Y12 secondary activation response is a key tipping point for excessive clot growth, leading to thrombosis. Current antiplatelet SOC non-aspirin monotherapies are mostly centered on P2Y12 inhibitors (clopidogrel, prasugrel, cangrelor, ticagrelor). Dual-antiplatelet therapies usually comprise P2Y12 inhibitors and aspirin. Aspirin, though beneficial and generally safe, has not solved clinical thrombosis and is now being used less, particularly for older patients who often have adverse effects. The unmet need addressed here is the well-documented and unsolved induction of clinical bleeding in patients on P2Y12 mono- or dual-therapies.

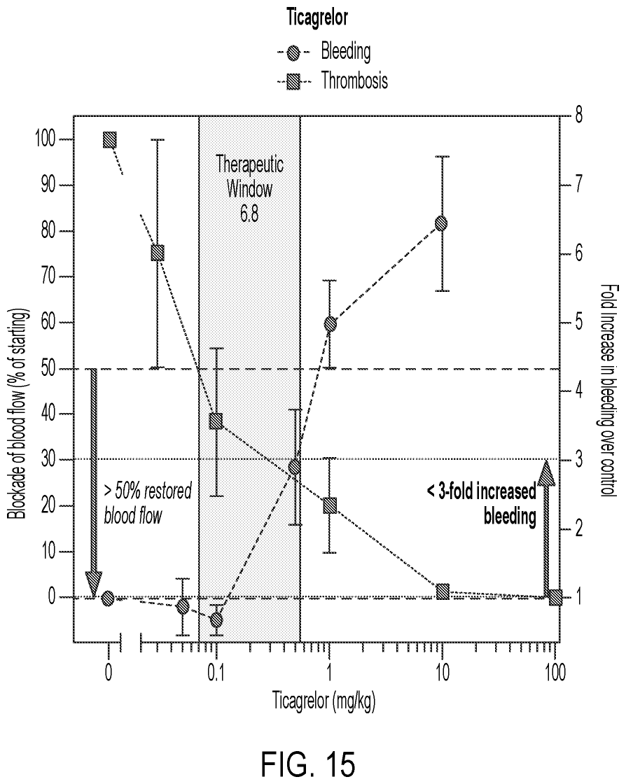

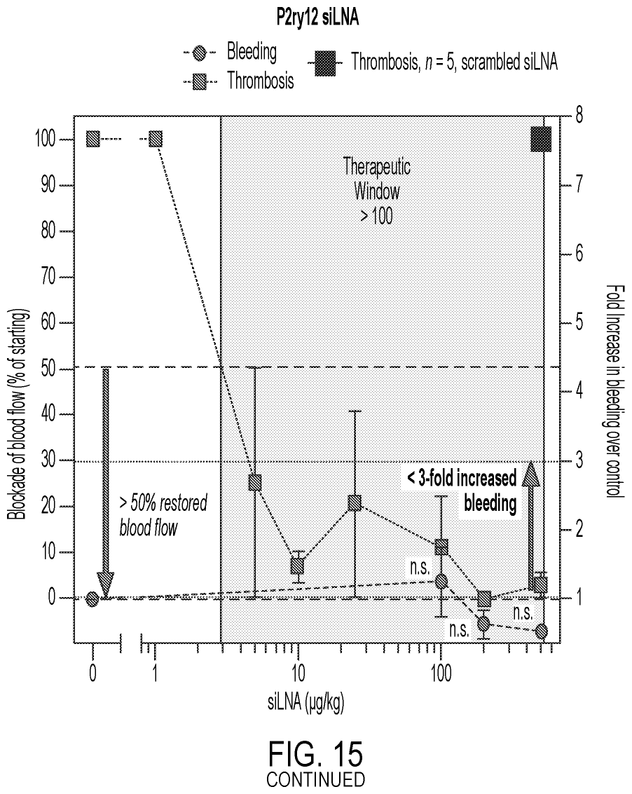

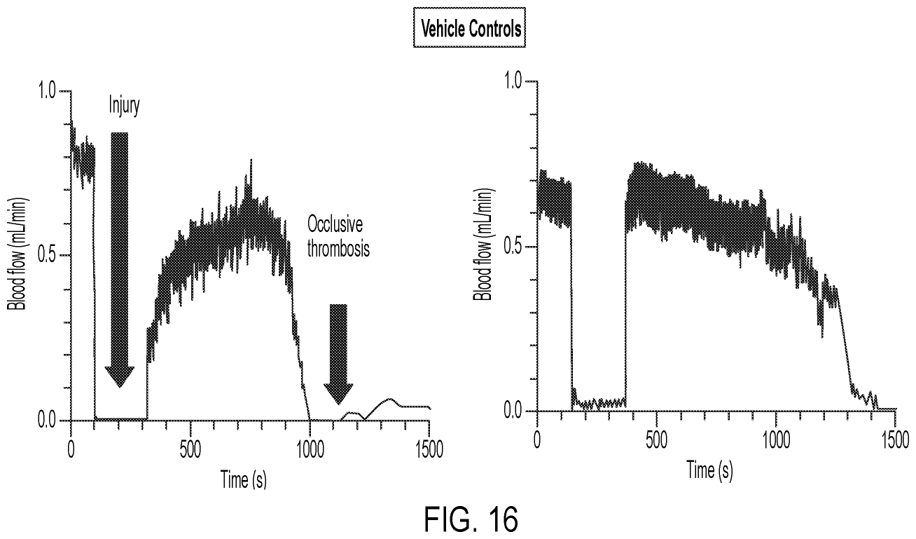

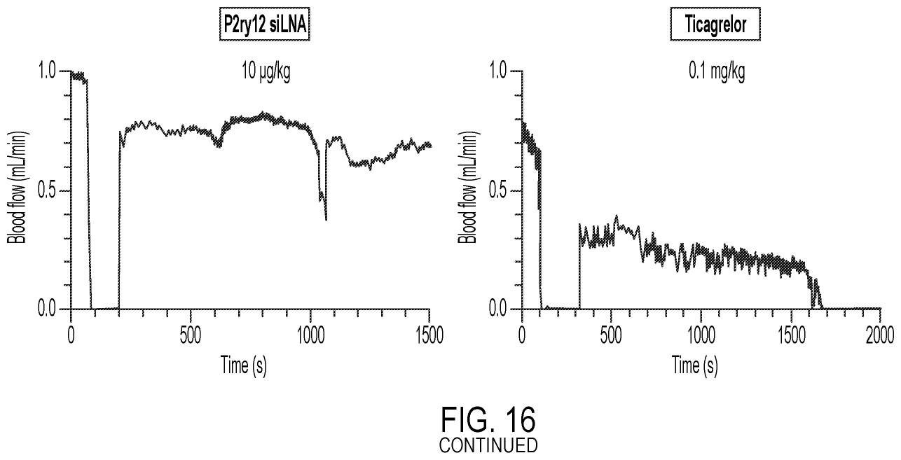

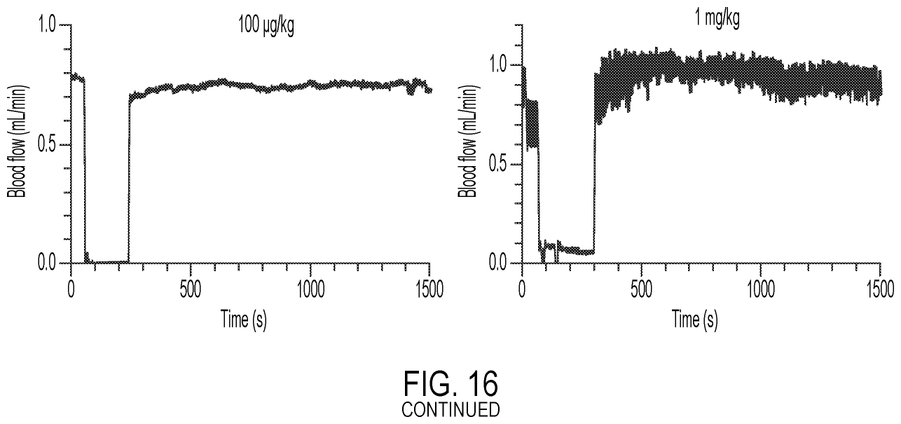

The data in FIG. 15 represent testing of the therapeutic window—the safe dose range which provides antithrombotic effects (therapeutic benefit) but does not cause excessive bleeding—for P2y12 siLNA, compared to that for P2Y12 antagonist ticagrelor, in mouse models. The P2y12 siLNA—as with all siLNAs—is designed to function as a siRNA once internalized in platelets and/or megakaryocytes. The siRNA function is to anchor specifically to the target mRNA (P2ry12, in this case) and prevent translation of the cognate protein (P2y12) via the cell's native RNA-induced silencing machinery.

As can be seen from FIG. 15, the TW for ticagrelor was determined to be 6.8 and that for P2ry12 siLNA was determined to be >100. Thus, the TW ratio, P2ry12 siLNA/ticagrelor >14-fold. The observed TW for ticagrelor in mice closely aligns with previously established TW in rats using similar approaches and reflects clinical observations in humans. The actual TW upper limit for P2ry12 siLNA remains unknown, as increased bleeding was not observed at the highest dose tested. Further, lower doses between 1 and 10 μg/kg may also provide therapeutic benefit (blockade of thrombosis). As control, thrombosis was assessed in mice injected with scrambled siLNA at the highest dose tested to day (0.5 mg/kg). Unlike P2ry12 siLNA, the scrambled control siLNA had no protective effect against thrombosis, as occlusion was observed in all the mice receiving the scrambled siLNA, similar to untreated mice subject to the thrombotic injury.

### Example 9: P2ry12 siLNA Destabilizes Thrombi and Prevents Thrombosis in a Manner Dependent on P2y12 Surface Densities

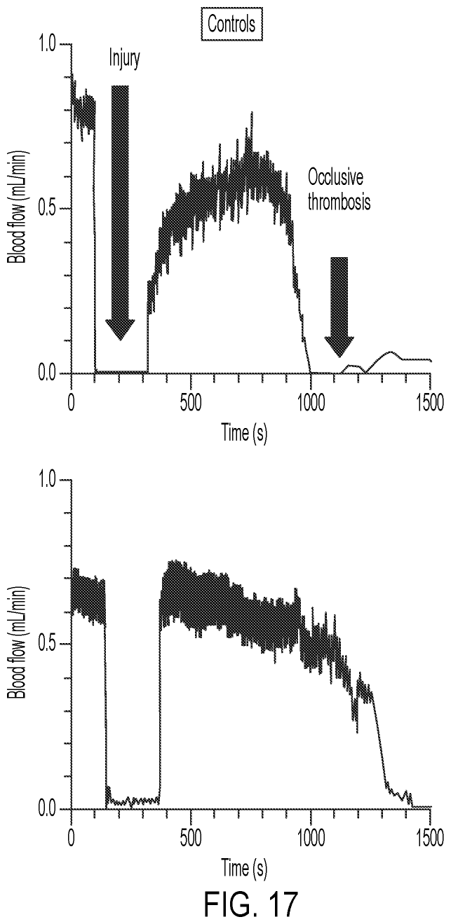

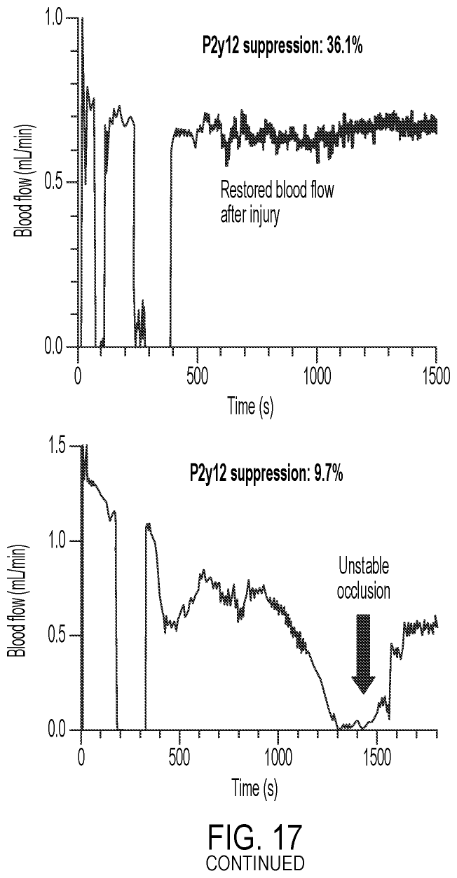

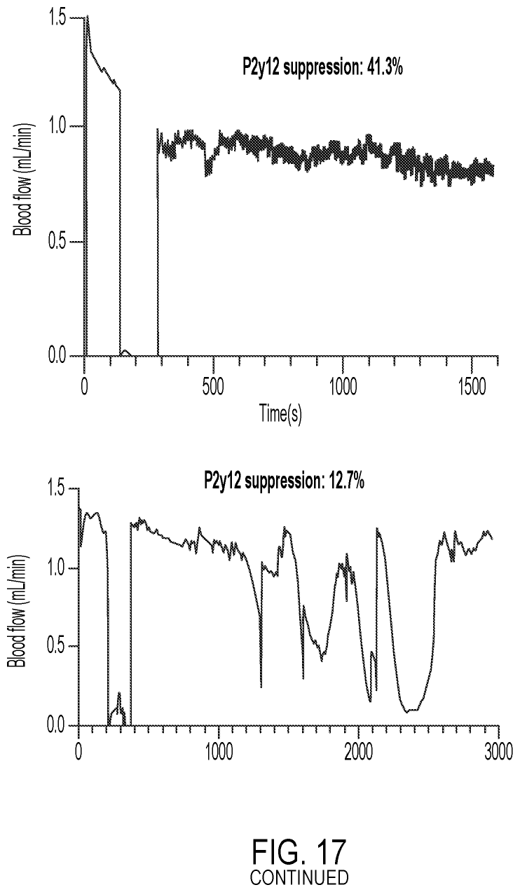

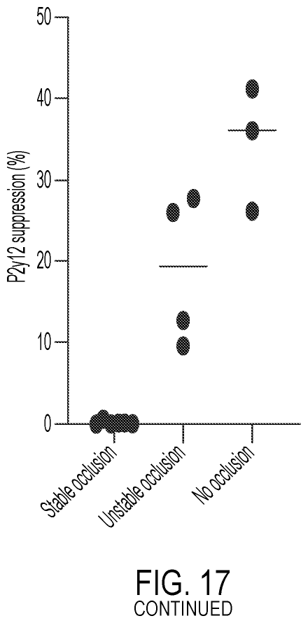

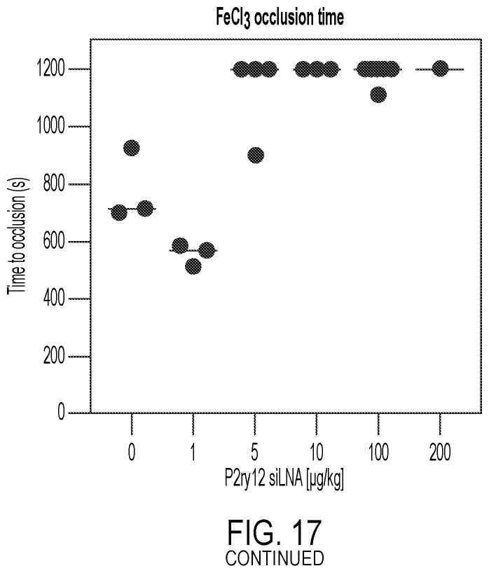

Blockade of thrombosis was tested as a function of P2y12 surface expression assessed by flow cytometry with fluorophore-conjugated P2y12 antibodies, at 24 hours after a single IV injection of a range of P2ry12 or scrambled siLNA. As in FIG. 17, it was observed that threshold levels for siLNA-mediated P2y12 suppression to destabilize thrombi and yield antithrombotic effects. ˜20% P2y12 suppression is sufficient to destabilize thrombi and restore blood flow, whereas ˜35-40% suppression appears sufficient to prevent formation of any occlusion despite vessel injury.

### Example 10: P2ry12 siLNA Normalizes High ADP Reactivity and P2y12 Levels Selectively in Newly Generated Platelets

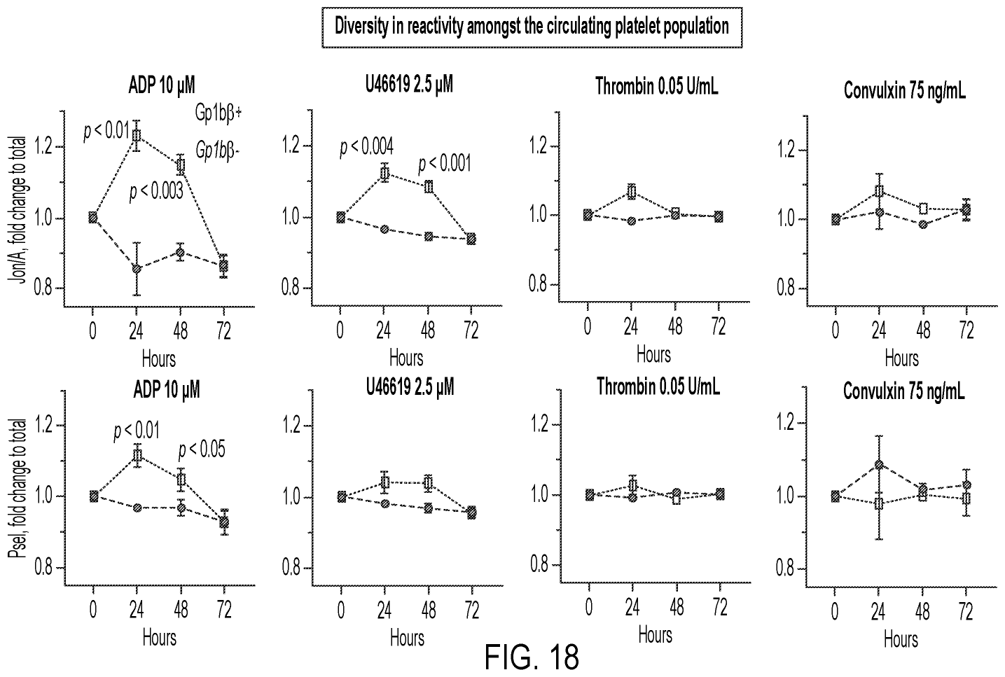

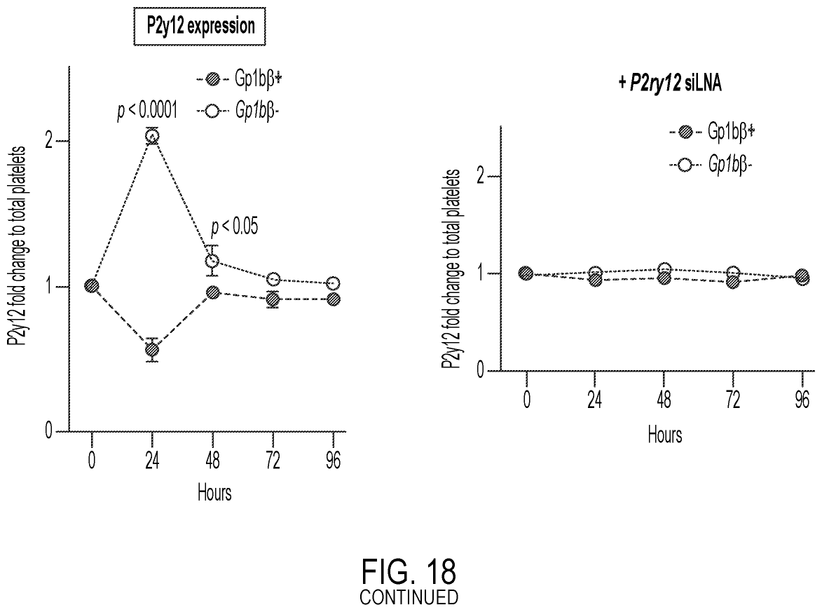

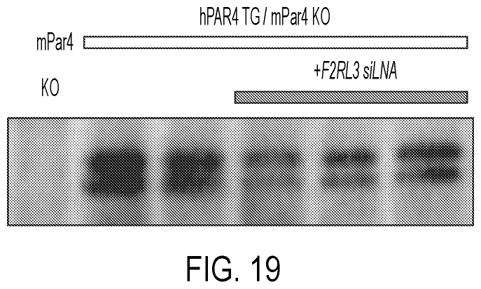

Platelet reactivity was monitored as a function of in vivo platelet lifespan. As shown in FIG. 18, newly generated platelets are hyperreactive to ADP (the physiological ligand for P2y12 receptor) as well as thromboxane to a lesser extent (another “secondary” agonist, blocked by aspirin), relative to mean reactivity of the total population, whereas thrombin and convulxin reactivity (major “primary” activation agonists) are similar across the lifespan. It was found that P2y12 surface densities are also highest in new platelets (FIG. 18, right) relative to the total population, and this high level of P2y12 decreases over time, indicating P2y12 synthesis mostly in megakaryocytes and/or newly generated platelets. P2y12 siLNA normalized this increase in the high-expressing subpopulation, but had no effect on the existing P2y12 levels, as would be predicted for siRNA-mediated gene silencing. Thus, P2ry12 siLNA reduces thrombosis by biased suppression of elevated P2y12 levels in younger platelets to normalize ADP reactivity, whereas older platelets with reduced P2y12 synthesis are refractory to siLNA-mediated silencing as the existing P2y12 protein is not targeted, and thus hemostasis is maintained.

Pulse-chase labeling with single infusion of Gp1bβ-X488 antibodies was used to monitor (FIG. 18, left) reactivity and (FIG. 18, right) P2y12 surface expression in ex vivo mouse platelets over their lifespan. Gp1bβ antibodies, conjugated to a fluorophore for tracking by flow cytometry, are injected into the blood stream where they specifically label platelets and no other cells, but these antibodies are inert and do no activate the platelets or cause them to be destroyed. The in vivo antibody labeling is saturated within one hour. Thus, when tracked by flow cytometry from blood samples, Gp1bβ+ platelets (fluorophore-positive, shown in solid line) represent the whole platelet population at the time of that pulse labeling. When platelets are extracted at later time points—in the above cases, at 24-hour intervals after the pulse label—the Gp1bβ-platelets (fluorophore-negative, shown in dotted line) represent newly generated platelets that were not subject to the pulse labeling at the “0”-hour time point. Over time, these Gp1bβ-platelets take over a larger and larger percentage of the total platelet pool, as the previously existing platelets are cleared over time (murine platelets have a ˜5-day lifespan). This is evident by the convergence of red and blue lines at later time points.

In FIG. 18, results are shown as relative integrin activation (top row) and α-granule secretion (bottom row)—two independent measures of platelet activation—or P2y12 levels, in new (Gp1bβ-, dotted line) and extant (Gp1bβ+, solid line) platelets compared to the total population. Top right, control; bottom right, 24 hrs post-P2ry12 siLNA injection. All ±s.e.m. n=3−6.

### Example 11: PAR4 as a Second (New) siLNA Target in Platelets

PAR4 is another platelet GPCR that is important for platelet activation. It is one of two major thrombin receptors on human platelets, and thrombin is a major agonist of so-called “primary” platelet activation that initiates clot formation. Mouse platelets only have Par4 as the sole thrombin receptor. PAR4 small molecule antagonists have been developed as putative antithrombotic drugs. It was considered whether PAR4 knockdown could yield benefit as an alternative antithrombotic target to P2Y12, as a single target, and potentially in conjunction with P2Y12 antagonists or P2Y12 siLNA. Unlike for P2y12/P2Y12, murine and human Par4/PAR4 genes are dissimilar enough that functional studies with clinical implications are best focused on the human gene if possible. Therefore, humanized PAR4 mice was developed harboring the human PAR4 transgene including all surrounding regulatory sequences, and deleted for murine Par4, backcrossed over many generations onto the WT C57B1/6J background. Platelet PAR4 has been previously studies, and it has been shown that the human PAR4 transgene, expressed on mouse platelets at similar levels as on human platelets, supports PAR4 functions in these mice that map onto human platelet physiology. Thus, these mice, referred to hereafter as ‘hPAR4’ mice, provide a unique resource for testing suppression of human PAR4 with a human-targeting siLNA that was designed and created (gene and siLNA name, F2RL3). IV injection of F2RL3 siLNA into these mice resulted in substantial suppression of human PAR4 in their platelets within 24 hrs.

Silencing of platelet hPAR4 yielded moderately reduced platelet reactivity to a receptor-specific peptide agonist, known as PAR4-activating peptide or PAR4-AP, which does not stimulate other PARs or receptors.

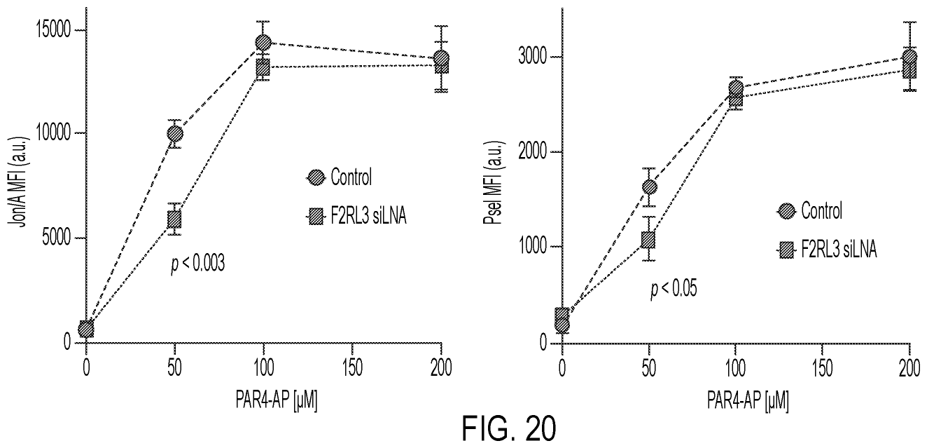

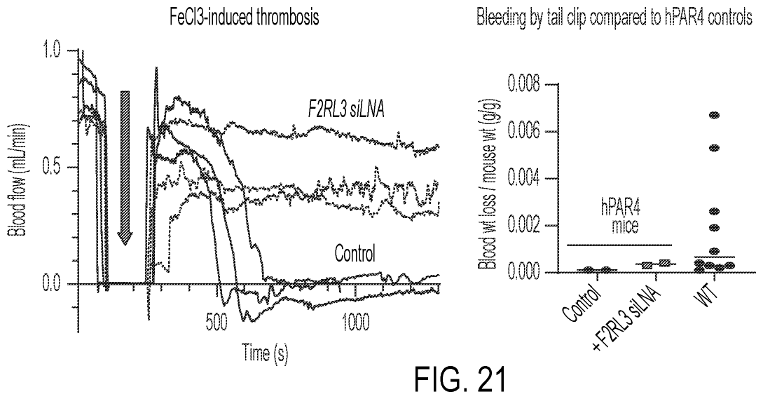

Since PAR4 is the sole reactive thrombin receptor on these platelets, it was predicted that moderate PAR4 suppression would be sufficient to achieve antithrombotic effects. As in FIG. 20, IV injection of naked F2RL3 siLNA—at the same dose that yielded moderate reduction in platelet reactivity to PAR4 agonist stimulation—was sufficient to block thrombosis in the FeCl₃ carotid arterial injury model at 24 hrs. However, despite the antithrombotic effect of F2RL3 siLNA—achieved at 500 g/kg which is the highest dose used to test P2y12 siLNA to date—increased bleeding in these mice compared to control hPAR4 or WT mice was not observed. The full therapeutic window for F2RL3 siLNA remains to be determined. These PAR4 data demonstrate that multiple siLNAs targeting distinct genes (mRNAs) are functional, as indicated by reduced target protein expression and expected functional effects.

### Example 12: SiLNA Targeting of Non-Receptor Cytosolic Proteins and Antibleeding/Pro-Hemostatic Effects

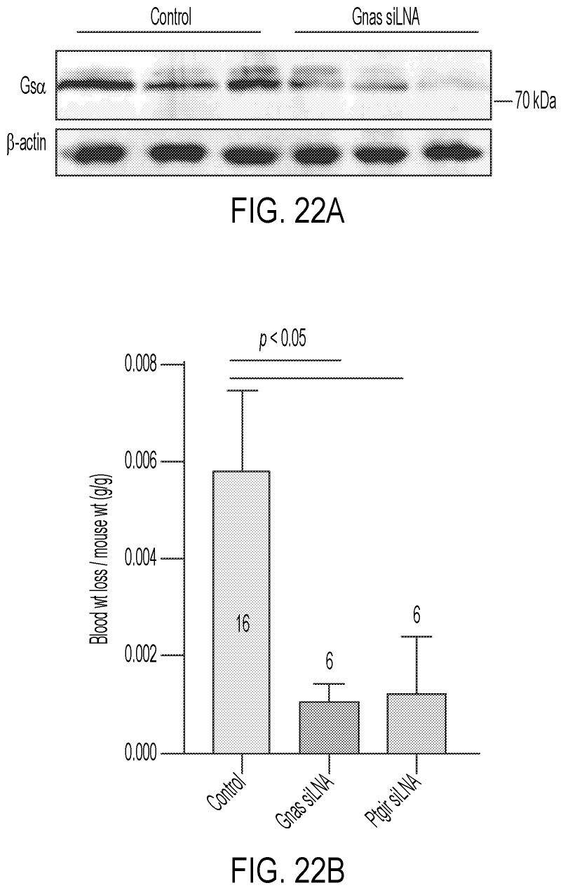

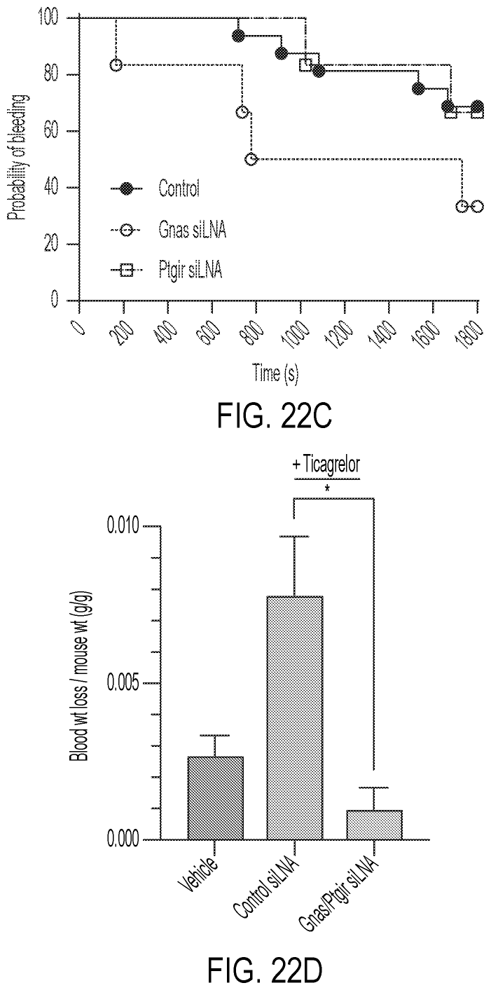

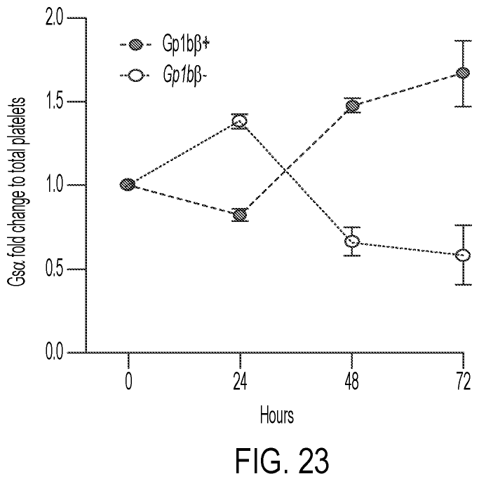

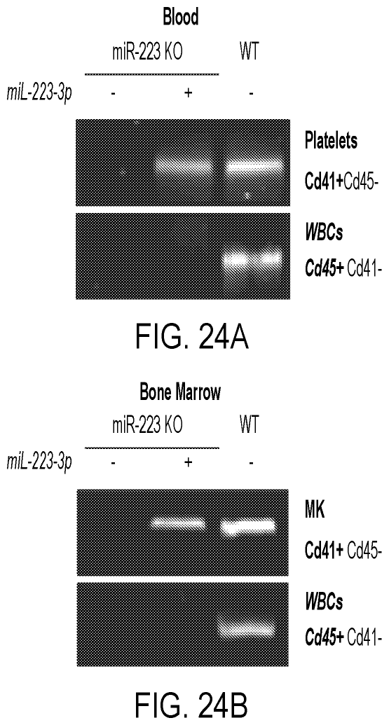

Gsα is a cytosolic small G protein that participates in platelet signaling. While P2y12 and PAR4 are distinct genes, their protein products are both members of the G protein-coupled receptor family. These data with respect to Gsα in FIG. 22 show that siLNAs silence expression of various types of protein targets. Of note, Gsα protein also shows distinct expression dynamics compared to P2y12 in circulating platelets. Gsα appears to be most highly expressed in older platelets. Thus, siLNAs normalize target protein expression across the platelet population, in a target-specific manner. In addition, the data in FIG. 22 demonstrate antibleeding/pro-hemostatic in vivo effects of siLNA-mediated suppression of endogenous negative regulatory proteins in platelets including Gsα (siLNA, Gnas) and IP (siLNA, Ptgir). The data in FIG. 22 further demonstrate rescue of acquired bleeding diathesis, by Gnas/Ptgir combined siLNAs in mice, treated acutely with medium dose ticagrelor which causes increased bleeding that was rescued by the siLNAs.

### Example 13: MiLNA/siLNA Platelet- and Megakaryocyte-Specific Uptake, as Evidenced by PCR

As an orthogonal approach to investigate megakaryocyte/platelet-selective in vivo uptake of naked mi/siLNAs, miR-223 knockout (KO) mice was used to monitor internalization of ectopic infused, naked miL-223-3p, by PCR in isolated cell populations that were FACSorted using specific markers. miR-223-3p was detected in all tested BM and blood cells in WT mice, but was absent in cells from miR-223 KO mice, as expected. Upon miL-223-3p infusion into miR-223 KO mice, it was observed that miR-223-3p is present in blood platelets and bone marrow megakaryocytes, but infused miLNA was not detected in blood or bone marrow white cells/leukocytes (WBCs, which includes Cd45+ WBC progenitors). As all these populations were single-cell sorted based on surface markers (and size, for megakaryocytes), heterotypic cell aggregates were eliminated. These results demonstrate that only megakaryocytes and platelets, but no other cells in hematopoietic tissues or blood, support the unique ability to internalize infused naked double-stranded miLNAs.

### Example 14: Broad-Spectrum Blood Cell Targeting by Anti-miRs

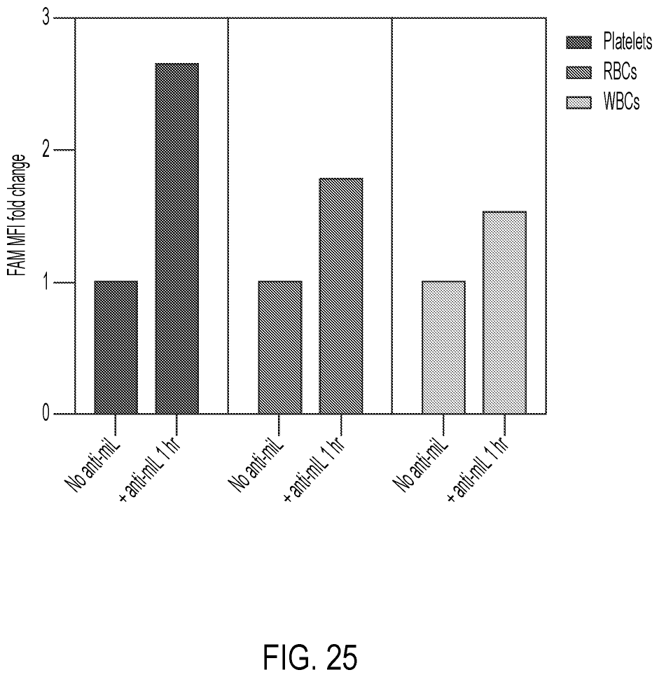

In contrast to platelet-/megakaryocyte-specific in vivo targeting by double-stranded miLNAs and siLNAs, it was found that a single-stranded 8-nucleotide anti-miR (ant-miR-223-3p)—comprised of locked nucleic acids (LNAs) modeling the McKenzie/Tsygankov anti-miR-148a and harboring a 3′-FAM fluorophore (anti-miL)—was internalized by blood platelets, and also internalized by red cells (RBCs) and white cells/leukocytes (WBCs) in vivo within 1 hr of intravenous administration in WT mice, as shown in FIG. 25. Of note, RBCs have substantially larger volumes than platelets, and WBCs are much larger than RBCs; these larger volumes relative to platelets account for apparently lower relative mean fluorescence intensities (per cell) observed. Hence, all blood cells show similar abilities to internalize anti-miRs modeled after anti-miR-148a. Thus, anti-miRs/antagomiRs do not harbor the unique property of platelet-/megakaryocyte-specific internalization and utilization that miLNAs and siLNAs do.

### Example 15: Long-Term Modulation of Platelet Reactivity by siLNA Using Subdermal Osmotic Pumps

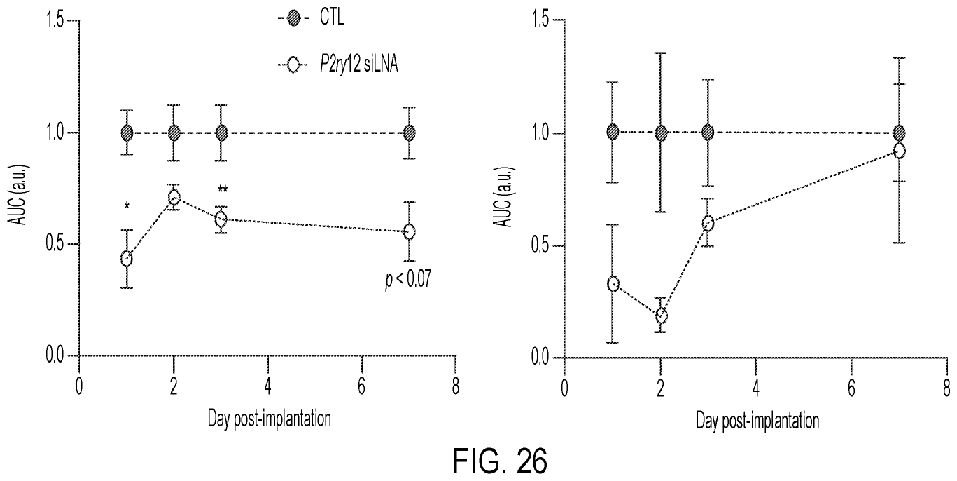

As shown in FIG. 26, platelet reactivity modulation can be achieved by subdermal administration, providing an alternate route to parental administration. Platelet ADP response was suppressed in mice harboring subdermal osmotic pumps with steady release of P2ry12 siLNA, demonstrating delivery of the siLNA payload to the bloodstream and to platelets, and to modulation of platelet function by subdermal administration of siLNAs.

### Enumerated Embodiments

The following exemplary embodiments are provided, the numbering of which is not to be construed as designating levels.

Embodiment 1 provides a method of selectively modulating gene expression of one or more target gene(s) in a cell of a subject, the method comprising administering to the subject a therapeutically effective amount of a composition comprising a naked RNA oligonucleotide,
wherein the cell is at least one selected from the group consisting of a megakaryocyte, a circulating platelet, and a platelet generated from a megakaryocyte transfected with the naked RNA oligonucleotide.

Embodiment 2 provide the method of embodiment 1, wherein the naked RNA oligonucleotide comprises a guide strand having a sequence selected from the guide strand sequences recited in Table 1 and a passenger strand having a sequence selected from the passenger strand sequences recited in Table 1.

Embodiment 3 provides the method of embodiment 1-2, wherein the naked RNA oligonucleotide is selected from the group consisting of an miRNA, an siRNA, and any combination thereof.

Embodiment 4 provides the method of embodiments 1-3, wherein the naked RNA oligonucleotide does not comprise any modified nucleotide/base.

Embodiment 5 provides the method of embodiments 1-4, wherein the transfection is performed without use of synthetic carrier or adjuvant.

Embodiment 6 provides the method of embodiments 1-5, wherein the naked RNA oligonucleotide optionally comprises at least one modification selected from the group consisting of a locked nucleic acid (LNA), a 2′-Fluorine base, and a 2′-O-methylated base.

Embodiment 7 provides the method of embodiments 1-6, wherein the naked RNA oligonucleotide is thermostable.

Embodiment 8 provides the method of embodiments 1-7, wherein the naked RNA oligonucleotide is non-immunogenic.

Embodiment 9 provides the method of embodiments 1-8, wherein the naked RNA oligonucleotide is resistant to cleavage by riboendonucleases (RNAases).

Embodiment 10 provides the method of embodiments 1-9, wherein the composition is administered intravenously.

Embodiment 11 provides the method of embodiments 1-10, wherein the administering alters megakaryocyte development, platelet production, megakaryocyte function, and/or platelet function.

Embodiment 12 provides the method of embodiments 1-11, wherein the subject is in need of at least one selected from the group consisting of:
i. an antiplatelet therapy,
ii. an anti-inflammatory therapy for treating thromboinflammation,
iii. a treatment for thrombocytopenia, thrombocytosis, and other pathological manifestations of disrupted megakaryocyte development,
iv. a platelet transfusion with platelets protected from platelet storage lesion,
v. a treatment for thrombosis,
vi. a treatment for acquired bleeding disorders, and
vii. a treatment for inherited bleeding disorders.

Embodiment 13 provides the method of embodiments 1-12, wherein the composition comprises a saline solution.

Embodiment 14 provides the method of embodiments 1-13, wherein the subject is a mammal.

Embodiment 15 provides the method of embodiments 1-14, wherein the mammal is a human.

Embodiment 16 provides a method of selectively modulating gene expression of one or more target genes in a cell, ex vivo or in vitro, the method comprising transfecting the cell with a naked RNA oligonucleotide, wherein the cell is at least one selected from a megakaryocyte and a platelet.

Embodiment 17 provide the method of embodiment 16, wherein the naked RNA oligonucleotide comprises a guide strand having a sequence selected from the guide strand sequences recited in Table 1 and a passenger strand having a sequence selected from the passenger strand sequences recited in Table 1.

Embodiment 18 provides the method of embodiments 16-17, wherein the cell is derived from at least one selected from the group consisting of a cell culture system, a concentrate, a cell suspension, a tissue homogenate, an organoid, a tissue, and an organ.

Embodiment 19 provides the method of embodiments 16-18, wherein the cell culture is a stem cell culture.

Embodiment 20 provides the method of embodiments 16-19, wherein the concentrate is a platelet storage concentrate containing autologous plasma.

Embodiment 21 provides the method of embodiments 16-20, wherein the transfection is performed without use of a synthetic carrier or an adjuvant.

Embodiment 22 provides the method of embodiments 16-21, wherein the naked RNA oligonucleotide is thermostable.

Embodiment 23 provides the method of embodiments 16-22, wherein the naked RNA oligonucleotide is non-immunogenic.

Embodiment 24 provides the method of embodiments 16-23, wherein the naked RNA oligonucleotide is resistant to cleavage by riboendonucleases (RNAases).

Embodiment 25 provides a method for delivering a naked RNA oligonucleotide to a target tissue and/or a cell of a subject, wherein the method comprises using a platelet transfected with the naked RNA oligonucleotide as a vehicle to deliver the naked RNA oligonucleotide to the target tissue and/or to the target cell.

Embodiment 26 provide the method of embodiment 25, wherein the naked RNA oligonucleotide comprises a guide strand having a sequence selected from the guide strand sequences recited in Table 1 and a passenger strand having a sequence selected from the passenger strand sequences recited in Table 1.

Embodiment 27 provides the method of embodiments 25-26, wherein the target cell is one selected from a tumor cell, a leukocyte and an inflammatory cell.

Embodiment 28 provides the method of embodiment 25-27, wherein the target tissue comprises an endothelial cell.

Embodiment 29 provides a composition for selectively modulating gene expression of one or more target genes in a cell of a subject, wherein the composition comprises a cell transfected with a naked RNA oligonucleotide, wherein the cell is at least one selected from the group consisting of a megakaryocyte, a circulating platelet, and a platelet generated from a megakaryocyte transfected with the naked RNA oligonucleotide.

Embodiment 30 provides the composition of embodiment 29, wherein the naked RNA oligonucleotide is suspended in an aqueous sterile saline solution.

Embodiment 31 provides a composition for selectively modulating gene expression of one or more target genes in a cell, ex vivo or in vitro, wherein the composition comprises a cell transfected with a naked RNA oligonucleotide, wherein the cell is at least one selected from the group consisting of a megakaryocyte and a platelet.

Embodiment 32 provides the composition of embodiment 31, wherein the cell is derived from one selected from the group consisting of a cell culture system, a concentrate, a cell suspension, a tissue homogenate, an organoid, a tissue, and an organ.

Embodiment 33 provides a kit comprising a composition having a naked RNA oligonucleotide for selectively modulating expression of at least one gene in a cell and an instructional material for use thereof, wherein the cell is at least one selected from the group consisting of a megakaryocyte, a platelet, and a platelet generated from a megakaryocyte transfected with the naked RNA oligonucleotide.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of selectively modulating gene expression of one or more target gene(s) in a cell of a subject, the method comprising administering to the subject a therapeutically effective amount of a composition comprising a naked RNA oligonucleotide,
wherein the cell is at least one selected from the group consisting of a megakaryocyte, a circulating platelet, and a platelet generated from a megakaryocyte transfected with the naked RNA oligonucleotide.

2. The method of claim 1, wherein the naked RNA oligonucleotide comprises a guide strand having a sequence selected from the guide strand sequences recited in Table 1 and a passenger strand having a sequence selected from the passenger strand sequences recited in Table 1.

3. The method of claim 1, wherein the naked RNA oligonucleotide is selected from the group consisting of an miRNA, an siRNA, and any combination thereof.

4. The method of claim 1, wherein the naked RNA oligonucleotide does not comprise any modified nucleotide/base.

5. The method of claim 1, wherein the transfection is performed without use of synthetic carrier or adjuvant.

6. The method of claim 1, wherein the naked RNA oligonucleotide optionally comprises at least one modification selected from the group consisting of a locked nucleic acid (LNA), a 2′-Fluorine base, and a 2′-O-methylated base.

7. The method of claim 6, wherein the naked RNA oligonucleotide is thermostable.

8. The method of claim 6, wherein the naked RNA oligonucleotide is non-immunogenic.

9. The method of claim 6, wherein the naked RNA oligonucleotide is resistant to cleavage by riboendonucleases (RNAases).

10. The method of claim 1, wherein the composition is administered intravenously.

11. The method of claim 1, wherein the administering alters megakaryocyte development, platelet production, megakaryocyte function, and/or platelet function.

12. The method of claim 1, wherein the subject is in need of at least one selected from the group consisting of:
i. an antiplatelet therapy,
ii. an anti-inflammatory therapy for treating thromboinflammation,
iii. a treatment for thrombocytopenia, thrombocytosis, and other pathological manifestations of disrupted megakaryocyte development,
iv. a platelet transfusion that requires protection from platelet storage lesion or transfusion is required due to the subject's platelet disorder,
v. a treatment for thrombosis,
vi. a treatment for acquired bleeding disorders, and
vii. a treatment for inherited bleeding disorders.

13. The method of claim 1, wherein the composition comprises a saline solution.

14. The method of claim 1, wherein the subject is a mammal.

15. The method of claim 14, wherein the mammal is a human.

16. A method of selectively modulating gene expression of one or more target genes in a cell, ex vivo or in vitro, the method comprising transfecting the cell with a naked RNA oligonucleotide, wherein the cell is at least one selected from a megakaryocyte and a platelet.

17. The method of claim 16, wherein the naked RNA oligonucleotide comprises a guide strand having a sequence selected from the guide strand sequences recited in Table 1 and a passenger strand having a sequence selected from the passenger strand sequences recited in Table 1.

18. The method of claim 16, wherein the cell is derived from at least one selected from the group consisting of a cell culture system, a concentrate, a cell suspension, a tissue homogenate, an organoid, a tissue, and an organ.

19. The method of claim 18, wherein the cell culture is a stem cell culture.

20. The method of claim 18, wherein the concentrate is a platelet storage concentrate containing autologous plasma.

21. The method of claim 16, wherein the transfecting comprises transfecting without use of a synthetic carrier or an adjuvant.

22. The method of claim 16, wherein the naked RNA oligonucleotide is thermostable.

23. The method of claim 16, wherein the naked RNA oligonucleotide is non-immunogenic.

24. The method of claim 16, wherein the naked RNA oligonucleotide is resistant to cleavage by riboendonucleases (RNAases).

25. A method for delivering a naked RNA oligonucleotide to a target tissue and/or a cell of a subject, wherein the method comprises using a platelet transfected with the naked RNA oligonucleotide as a vehicle to deliver the naked RNA oligonucleotide to the target tissue and/or to the target cell.

26. The method of claim 25, wherein the naked RNA oligonucleotide comprises a guide strand having a sequence selected from the guide strand sequences recited in Table 1 and a passenger strand having a sequence selected from the passenger strand sequences recited in Table 1.

27. The method of claim 25, wherein the target cell is one selected from a tumor cell, a leukocyte or an inflammatory cell.

28. The method of claim 25, wherein the target tissue comprises an endothelial cell.

29. A composition for selectively modulating gene expression of one or more target genes in a cell of a subject, wherein the composition comprises a cell transfected with a naked RNA oligonucleotide, wherein the cell is at least one selected from the group consisting of a megakaryocyte, a circulating platelet, and a platelet generated from a megakaryocyte transfected with the naked RNA oligonucleotide.

30. The composition of claim 29, wherein the naked RNA oligonucleotide is suspended in an aqueous sterile saline solution.

31. A composition for selectively modulating gene expression of one or more target genes in a cell, ex vivo or in vitro, wherein the composition comprises a cell transfected with a naked RNA oligonucleotide, wherein the cell is at least one selected from the group consisting of a megakaryocyte and a platelet.

32. The composition of claim 31, wherein the cell is derived from one selected from the group consisting of a cell culture system, a concentrate, a cell suspension, a tissue homogenate, an organoid, a tissue, and an organ.

33. A kit comprising a composition having a naked RNA oligonucleotide for selectively modulating expression of at least one gene in a cell and an instructional material for use thereof, wherein the cell is at least one selected from the group consisting of a megakaryocyte, a platelet, and a platelet generated from a megakaryocyte transfected with the naked RNA oligonucleotide.


SEQUENCE LISTING (124 sequences)

SEQ ID NO: 1 - RNA, 21 nt, synthetic construct
  source 1..21: mol_type = other RNA
agagtaagaacatgaatgccc

SEQ ID NO: 2 - RNA, 21 nt, synthetic construct
  source 1..21: mol_type = other RNA
agaaagaggccagtatgtggt

SEQ ID NO: 3 - RNA, 21 nt, synthetic construct
  source 1..21: mol_type = other RNA
aatgagattcccattgtggaa

SEQ ID NO: 4 - RNA, 21 nt, synthetic construct
  source 1..21: mol_type = other RNA
tgaaagtgccctttagttctg

SEQ ID NO: 5 - RNA, 21 nt, synthetic construct
  source 1..21: mol_type = other RNA
taaagagagaatatcgtagtc

SEQ ID NO: 6 - RNA, 21 nt, synthetic construct
  source 1..21: mol_type = other RNA
aaatgacagagtgaaatggca

SEQ ID NO: 7 - RNA, 21 nt, synthetic construct
  source 1..21: mol_type = other RNA
taacaaagcaatgataaaccc

SEQ ID NO: 8 - RNA, 21 nt, synthetic construct
  source 1..21: mol_type = other RNA
atgaaactcaggtttctgcac

SEQ ID NO: 9 - RNA, 21 nt, synthetic construct
  source 1..21: mol_type = other RNA
tgtatgagtcgaagatctccc

SEQ ID NO: 10 - RNA, 21 nt, synthetic construct
  source 1..21: mol_type = other RNA
taatgtgagggaacttcagga

SEQ ID NO: 11 - RNA, 21 nt, synthetic construct
  source 1..21: mol_type = other RNA
tttttctctagcttcttgcag

SEQ ID NO: 12 - RNA, 21 nt, synthetic construct
  source 1..21: mol_type = other RNA
atcttcttcagcttcagcgct

SEQ ID NO: 13 - RNA, 21 nt, synthetic construct
  source 1..21: mol_type = other RNA
atacagatgtgataactggcg

SEQ ID NO: 14 - RNA, 21 nt, synthetic construct
  source 1..21: mol_type = other RNA
aacaaagtcaattcttcgctg

SEQ ID NO: 15 - RNA, 21 nt, synthetic construct
  source 1..21: mol_type = other RNA
aagcaaatgatgaagatgcag

SEQ ID NO: 16 - RNA, 21 nt, synthetic construct
  source 1..21: mol_type = other RNA
ttcatcagcagcatggtggag

SEQ ID NO: 17 - RNA, 21 nt, synthetic construct
  source 1..21: mol_type = other RNA
tcattttccacgtaaatgcgg

SEQ ID NO: 18 - RNA, 21 nt, synthetic construct
  source 1..21: mol_type = other RNA
ataagcatcaacaatgagctg

SEQ ID NO: 19 - RNA, 21 nt, synthetic construct
  source 1..21: mol_type = other RNA
tcaatgtcggccacatagttc

SEQ ID NO: 20 - RNA, 21 nt, synthetic construct
  source 1..21: mol_type = other RNA
tcttcttcaaagatgaaggga

SEQ ID NO: 21 - RNA, 21 nt, synthetic construct
  source 1..21: mol_type = other RNA
actttctgtacttcttacggt

SEQ ID NO: 22 - RNA, 21 nt, synthetic construct
  source 1..21: mol_type = other RNA
acaatcaccagtttcttccgg

SEQ ID NO: 23 - RNA, 21 nt, synthetic construct
  source 1..21: mol_type = other RNA
aatatccagggagagattccg

SEQ ID NO: 24 - RNA, 21 nt, synthetic construct
  source 1..21: mol_type = other RNA
tcaaagtgggacttcttgctg

SEQ ID NO: 25 - RNA, 21 nt, synthetic construct
  source 1..21: mol_type = other RNA
aaagtagtcctcaatcttcga

SEQ ID NO: 26 - RNA, 21 nt, synthetic construct
  source 1..21: mol_type = other RNA
tgagcatggacgccaggccga

SEQ ID NO: 27 - RNA, 21 nt, synthetic construct
  source 1..21: mol_type = other RNA
aaatgatccatttccaatcac

SEQ ID NO: 28 - RNA, 21 nt, synthetic construct
  source 1..21: mol_type = other RNA
agacgcggcccaagggctcgt

SEQ ID NO: 29 - RNA, 22 nt, synthetic construct
  source 1..22: mol_type = other RNA
tgtcagtttgtcaaatacccca

SEQ ID NO: 30 - RNA, 23 nt, synthetic construct
  source 1..23: mol_type = other RNA
gtccagttttcccaggaatccct

SEQ ID NO: 31 - RNA, 22 nt, synthetic construct
  source 1..22: mol_type = other RNA
tctcccaacccttgtaccagtg

SEQ ID NO: 32 - RNA, 22 nt, synthetic construct
  source 1..22: mol_type = other RNA
tccctgagaccctaacttgtga

SEQ ID NO: 33 - RNA, 21 nt, synthetic construct
  source 1..21: mol_type = other RNA
gcattcatgttcttactcttt

SEQ ID NO: 34 - RNA, 21 nt, synthetic construct
  source 1..21: mol_type = other RNA
cacatactggcctctttcttt

SEQ ID NO: 35 - RNA, 21 nt, synthetic construct
  source 1..21: mol_type = other RNA
ccacaatgggaatctcatttt

SEQ ID NO: 36 - RNA, 21 nt, synthetic construct
  source 1..21: mol_type = other RNA
gaactaaagggcactttcatt

SEQ ID NO: 37 - RNA, 21 nt, synthetic construct
  source 1..21: mol_type = other RNA
ctacgatattctctctttatt

SEQ ID NO: 38 - RNA, 21 nt, synthetic construct
  source 1..21: mol_type = other RNA
ccatttcactctgtcattttt

SEQ ID NO: 39 - RNA, 21 nt, synthetic construct
  source 1..21: mol_type = other RNA
gtttatcattgctttgttatt

SEQ ID NO: 40 - RNA, 21 nt, synthetic construct
  source 1..21: mol_type = other RNA
gcagaaacctgagtttcattt

SEQ ID NO: 41 - RNA, 21 nt, synthetic construct
  source 1..21: mol_type = other RNA
gagatcttcgactcatacatt

SEQ ID NO: 42 - RNA, 21 nt, synthetic construct
  source 1..21: mol_type = other RNA
ctgaagttccctcacattatt

SEQ ID NO: 43 - RNA, 21 nt, synthetic construct
  source 1..21: mol_type = other RNA
gcaagaagctagagaaaaatt

SEQ ID NO: 44 - RNA, 21 nt, synthetic construct
  source 1..21: mol_type = other RNA
cgctgaagctgaagaagattt

SEQ ID NO: 45 - RNA, 21 nt, synthetic construct
  source 1..21: mol_type = other RNA
ccagttatcacatctgtattt

SEQ ID NO: 46 - RNA, 21 nt, synthetic construct
  source 1..21: mol_type = other RNA
gcgaagaattgactttgtttt

SEQ ID NO: 47 - RNA, 21 nt, synthetic construct
  source 1..21: mol_type = other RNA
gcatcttcatcatttgctttt

SEQ ID NO: 48 - RNA, 21 nt, synthetic construct
  source 1..21: mol_type = other RNA
ccaccatgctgctgatgaatt

SEQ ID NO: 49 - RNA, 21 nt, synthetic construct
  source 1..21: mol_type = other RNA
gcatttacgtggaaaatgatt

SEQ ID NO: 50 - RNA, 21 nt, synthetic construct
  source 1..21: mol_type = other RNA
gctcattgttgatgcttattt

SEQ ID NO: 51 - RNA, 21 nt, synthetic construct
  source 1..21: mol_type = other RNA
actatgtggccgacattgatt

SEQ ID NO: 52 - RNA, 21 nt, synthetic construct
  source 1..21: mol_type = other RNA
ccttcatctttgaagaagatt

SEQ ID NO: 53 - RNA, 21 nt, synthetic construct
  source 1..21: mol_type = other RNA
cgtaagaagtacagaaagttt

SEQ ID NO: 54 - RNA, 21 nt, synthetic construct
  source 1..21: mol_type = other RNA
ggaagaaactggtgattgttt

SEQ ID NO: 55 - RNA, 21 nt, synthetic construct
  source 1..21: mol_type = other RNA
gaatctctccctggatatttt

SEQ ID NO: 56 - RNA, 21 nt, synthetic construct
  source 1..21: mol_type = other RNA
gcaagaagtcccactttgatt

SEQ ID NO: 57 - RNA, 21 nt, synthetic construct
  source 1..21: mol_type = other RNA
gaagattgaggactacttttt

SEQ ID NO: 58 - RNA, 21 nt, synthetic construct
  source 1..21: mol_type = other RNA
ggcctggcgtccatgctcatt

SEQ ID NO: 59 - RNA, 21 nt, synthetic construct
  source 1..21: mol_type = other RNA
gattggaaatggatcattttt

SEQ ID NO: 60 - RNA, 21 nt, synthetic construct
  source 1..21: mol_type = other RNA
gagcccttgggccgcgtcttt

SEQ ID NO: 61 - RNA, 22 nt, synthetic construct
  source 1..22: mol_type = other RNA
gggtatttgacaaactgacatt

SEQ ID NO: 62 - RNA, 21 nt, synthetic construct
  source 1..21: mol_type = other RNA
ggattcctgggaaaactggtt

SEQ ID NO: 63 - RNA, 22 nt, synthetic construct
  source 1..22: mol_type = other RNA
ctggtacaagggttgggagatt

SEQ ID NO: 64 - RNA, 22 nt, synthetic construct
  source 1..22: mol_type = other RNA
acaagttagggtctcagggatt

SEQ ID NO: 65 - RNA, 21 nt, synthetic construct
  source 1..21: mol_type = other RNA
agaatcatgagaagatcagaa

SEQ ID NO: 66 - RNA, 21 nt, synthetic construct
  source 1..21: mol_type = other RNA
aagaaagaggccagtatgtgg

SEQ ID NO: 67 - RNA, 21 nt, synthetic construct
  source 1..21: mol_type = other RNA
tacattctgagacatttgggt

SEQ ID NO: 68 - RNA, 21 nt, synthetic construct
  source 1..21: mol_type = other RNA
aatatctccagtttgtgtcac

SEQ ID NO: 69 - RNA, 21 nt, synthetic construct
  source 1..21: mol_type = other RNA
aataaccacacacttttggag

SEQ ID NO: 70 - RNA, 21 nt, synthetic construct
  source 1..21: mol_type = other RNA
tcttcatacagcatacatggc

SEQ ID NO: 71 - RNA, 21 nt, synthetic construct
  source 1..21: mol_type = other RNA
ttccagaagattctccaggga

SEQ ID NO: 72 - RNA, 21 nt, synthetic construct
  source 1..21: mol_type = other RNA
tgtataactgtcttgttccat

SEQ ID NO: 73 - RNA, 21 nt, synthetic construct
  source 1..21: mol_type = other RNA
aaatctcttcgatgtatggct

SEQ ID NO: 74 - RNA, 21 nt, synthetic construct
  source 1..21: mol_type = other RNA
ttcatgttcctgaagaagggg

SEQ ID NO: 75 - RNA, 21 nt, synthetic construct
  source 1..21: mol_type = other RNA
tcttgatccgctttttctcta

SEQ ID NO: 76 - RNA, 21 nt, synthetic construct
  source 1..21: mol_type = other RNA
tttccttccattcttcagcgt

SEQ ID NO: 77 - RNA, 21 nt, synthetic construct
  source 1..21: mol_type = other RNA
aattccttctacgcatttccc

SEQ ID NO: 78 - RNA, 21 nt, synthetic construct
  source 1..21: mol_type = other RNA
tcttgttgcttctaactgcag

SEQ ID NO: 79 - RNA, 21 nt, synthetic construct
  source 1..21: mol_type = other RNA
tttcattgagcacatcatgac

SEQ ID NO: 80 - RNA, 21 nt, synthetic construct
  source 1..21: mol_type = other RNA
atacatgtgaccatagagtgc

SEQ ID NO: 81 - RNA, 21 nt, synthetic construct
  source 1..21: mol_type = other RNA
actgaatgcccaaaatacgac

SEQ ID NO: 82 - RNA, 21 nt, synthetic construct
  source 1..21: mol_type = other RNA
tgtttgtagccaaacatgggc

SEQ ID NO: 83 - RNA, 21 nt, synthetic construct
  source 1..21: mol_type = other RNA
tagttctcgaagacggtgggc

SEQ ID NO: 84 - RNA, 21 nt, synthetic construct
  source 1..21: mol_type = other RNA
tcttgttgaagacaaatcgct

SEQ ID NO: 85 - RNA, 21 nt, synthetic construct
  source 1..21: mol_type = other RNA
agattgatctcaatcttgggg

SEQ ID NO: 86 - RNA, 21 nt, synthetic construct
  source 1..21: mol_type = other RNA
aacaatcaccagtttcttccg

SEQ ID NO: 87 - RNA, 21 nt, synthetic construct
  source 1..21: mol_type = other RNA
catagacactgattttggcca

SEQ ID NO: 88 - RNA, 21 nt, synthetic construct
  source 1..21: mol_type = other RNA
tcatgaacccttctttaagca

SEQ ID NO: 89 - RNA, 21 nt, synthetic construct
  source 1..21: mol_type = other RNA
actcaaatggcatagcttctt

SEQ ID NO: 90 - RNA, 21 nt, synthetic construct
  source 1..21: mol_type = other RNA
aagaggatgagcatggacgcc

SEQ ID NO: 91 - RNA, 21 nt, synthetic construct
  source 1..21: mol_type = other RNA
ctgatcttctcatgattcttt

SEQ ID NO: 92 - RNA, 21 nt, synthetic construct
  source 1..21: mol_type = other RNA
acatactggcctctttctttt

SEQ ID NO: 93 - RNA, 21 nt, synthetic construct
  source 1..21: mol_type = other RNA
ccaaatgtctcagaatgtatt

SEQ ID NO: 94 - RNA, 21 nt, synthetic construct
  source 1..21: mol_type = other RNA
gacacaaactggagatatttt

SEQ ID NO: 95 - RNA, 21 nt, synthetic construct
  source 1..21: mol_type = other RNA
ccaaaagtgtgtggttatttt

SEQ ID NO: 96 - RNA, 21 nt, synthetic construct
  source 1..21: mol_type = other RNA
catgtatgctgtatgaagatt

SEQ ID NO: 97 - RNA, 21 nt, synthetic construct
  source 1..21: mol_type = other RNA
cctggagaatcttctggaatt

SEQ ID NO: 98 - RNA, 21 nt, synthetic construct
  source 1..21: mol_type = other RNA
ggaacaagacagttatacatt

SEQ ID NO: 99 - RNA, 21 nt, synthetic construct
  source 1..21: mol_type = other RNA
ccatacatcgaagagattttt

SEQ ID NO: 100 - RNA, 21 nt, synthetic construct
  source 1..21: mol_type = other RNA
ccttcttcaggaacatgaatt

SEQ ID NO: 101 - RNA, 21 nt, synthetic construct
  source 1..21: mol_type = other RNA
gagaaaaagcggatcaagatt

SEQ ID NO: 102 - RNA, 21 nt, synthetic construct
  source 1..21: mol_type = other RNA
gctgaagaatggaaggaaatt

SEQ ID NO: 103 - RNA, 21 nt, synthetic construct
  source 1..21: mol_type = other RNA
gaaatgcgtagaaggaatttt

SEQ ID NO: 104 - RNA, 21 nt, synthetic construct
  source 1..21: mol_type = other RNA
gcagttagaagcaacaagatt

SEQ ID NO: 105 - RNA, 21 nt, synthetic construct
  source 1..21: mol_type = other RNA
catgatgtgctcaatgaaatt

SEQ ID NO: 106 - RNA, 21 nt, synthetic construct
  source 1..21: mol_type = other RNA
actctatggtcacatgtattt

SEQ ID NO: 107 - RNA, 21 nt, synthetic construct
  source 1..21: mol_type = other RNA
cgtattttgggcattcagttt

SEQ ID NO: 108 - RNA, 21 nt, synthetic construct
  source 1..21: mol_type = other RNA
ccatgtttggctacaaacatt

SEQ ID NO: 109 - RNA, 21 nt, synthetic construct
  source 1..21: mol_type = other RNA
ccaccgtcttcgagaactatt

SEQ ID NO: 110 - RNA, 21 nt, synthetic construct
  source 1..21: mol_type = other RNA
cgatttgtcttcaacaagatt

SEQ ID NO: 111 - RNA, 21 nt, synthetic construct
  source 1..21: mol_type = other RNA
ccaagattgagatcaatcttt

SEQ ID NO: 112 - RNA, 21 nt, synthetic construct
  source 1..21: mol_type = other RNA
gaagaaactggtgattgtttt

SEQ ID NO: 113 - RNA, 21 nt, synthetic construct
  source 1..21: mol_type = other RNA
gccaaaatcagtgtctatgtt

SEQ ID NO: 114 - RNA, 21 nt, synthetic construct
  source 1..21: mol_type = other RNA
cttaaagaagggttcatgatt

SEQ ID NO: 115 - RNA, 21 nt, synthetic construct
  source 1..21: mol_type = other RNA
gaagctatgccatttgagttt

SEQ ID NO: 116 - RNA, 21 nt, synthetic construct
  source 1..21: mol_type = other RNA
cgtccatgctcatcctctttt

SEQ ID NO: 117 - RNA, 19 nt, synthetic construct
  source 1..19: mol_type = other RNA
ctcgtcattccgaaggtcc

SEQ ID NO: 118 - RNA, 18 nt, synthetic construct
  source 1..18: mol_type = other RNA
ccttcggaatgacgagtt

SEQ ID NO: 119 - RNA, 19 nt, synthetic construct
  source 1..19: mol_type = other RNA
ctcactccatctttggtct

SEQ ID NO: 120 - RNA, 18 nt, synthetic construct
  source 1..18: mol_type = other RNA
ccaaagatggagtgagtt

SEQ ID NO: 121 - RNA, 24 nt, synthetic construct
  source 1..24: mol_type = other RNA
tcacaacctcctagaaagagtaga

SEQ ID NO: 122 - RNA, 24 nt, synthetic construct
  source 1..24: mol_type = other RNA
tactctttctaggaggttgtgatt

SEQ ID NO: 123 - RNA, 21 nt, synthetic construct
  source 1..21: mol_type = other RNA
agaatcattagtagatcagag

SEQ ID NO: 124 - RNA, 21 nt, synthetic construct
  source 1..21: mol_type = other RNA
ctgatctactaatgattcttt